(19) United States
(12) Patent Application Publication
(10) Pub. No.: US 2024/0168036 A1
(43) Pub. Date: May 23, 2024

(54) IDENTIFICATION AND QUANTITATION OF RESIDUAL HOST CELL PROTEINS IN PROTEIN SAMPLES

(71) Applicants: GENENTECH, INC., South San Francisco, CA (US); HOFFMANN-LA ROCHE INC., Little Falls, NJ (US)

(72) Inventors: Jia Guo, Los Altos, CA (US); Regina Kufer, Munich (DE); Delia Li, Cupertino, CA (US); Feng Yang, Foster City, CA (US); Inn Huam Yuk, Burlingame, CA (US); Jinhua Zhang, Foster City, CA (US); Lance J. Cadang, Hercules, CA (US); Lu Dai, San Mateo, CA (US)

(73) Assignees: GENENTECH, INC., South San Francisco, CA (US); HOFFMANN-LA ROCHE INC., Little Falls, NJ (US)

(21) Appl. No.: 18/526,020

(22) Filed: Dec. 1, 2023

Related U.S. Application Data
(63) Continuation of application No. PCT/US2022/032099, filed on Jun. 3, 2022.
(60) Provisional application No. 63/197,052, filed on Jun. 4, 2021.

(51) Int. Cl. G01N 33/68; G01N 1/40; G01N 30/72; G01N 30/88

(52) U.S. Cl. CPC G01N 33/6848; G01N 1/4044; G01N 30/7233; G01N 30/88; G01N 2030/8831

(57) ABSTRACT

The present disclosure relates to highly sensitive methods for determining the identity and quantity of one or more proteins in a sample. For example, the present disclosure provides methods for the highly sensitive identification and quantitation of residual host cell proteins in protein samples and can be adapted to identify and quantify proteins in either a targeted or a target-agnostic manner and can be modified to achieve a range of sensitivities appropriate for distinct use cases.

### CROSS-REFERENCE TO RELATED APPLICATIONS

This application is a continuation of PCT Application No. PCT/US22/032099, filed Jun. 3, 2022, which claims priority to U.S. Provisional Application No. 63/197,052, filed on Jun. 4, 2021, the contents of both of which are incorporated by reference in their entireties, and to which priority is claimed.

### SEQUENCE LISTING

This application contains a Sequence Listing which has been submitted electronically in XML format and is hereby incorporated by reference in its entirety. Said sequence listing copy, created on Nov. 22, 2023, is named “00B206.1393_ST26.xml” and is 21,204 bytes in size.

### FIELD OF INVENTION

The present disclosure relates to highly sensitive methods for determining the identity and quantity of one or more proteins in a sample. For example, the present disclosure provides methods for the highly sensitive identification and quantitation of residual host cell proteins in protein samples and can be adapted to identify and quantify proteins in either a targeted or a target-agnostic manner and can be modified to achieve a range of sensitivities appropriate for distinct use cases.

### BACKGROUND

The large-scale, economic purification of polypeptides is increasingly an important problem for the biotechnology industry. Generally, polypeptides are produced by cell culture, using either mammalian or bacterial cell lines engineered to produce the polypeptide of interest by insertion of a recombinant plasmid containing the gene for that polypeptide. Since the cell lines used are living organisms, it is desirable to separate the polypeptide of interest from a mixture of compounds fed to the cells as well as endogenous proteins made by the cells themselves (host cell proteins or “HCPs”).

The separation of the polypeptide of interest from HCPs is usually achieved using a combination of different chromatography techniques. While such sophisticated separation strategies employed in the biotechnology industry are capable of highly efficient removal of HCPs to thereby provide purified preparations of polypeptides of interest, the quantification of extremely low levels of HCPs remains a highly desirable goal. For example, residual hydrolytic enzymes (“hydrolases”) present in final product compositions, e.g. ultrafiltration and diafiltration (UF/DF) pools, drug substance (DS) and/or drug product (DP), can cause polysorbate (PS) degradation in the drug product and lead to visual particle (VP) and sub-visible particle (SVP) concerns. This is the case even when those hydrolases are present at extremely low levels. Thus, having effective strategies to identify and quantitate low levels of HCPs is particularly useful for process development or investigations to support biologics manufacturing.

While existing solutions, such as fatty acid by mass spectrometry (“FAMS”) analyses or activity-based assays have been effective in addressing certain aspects of PS degradation, the root causes, i.e., the hydrolases that contribute to the PS20 degradation in a final product are largely unknown. This lack of knowledge around the specific identity of the residual HCPs present a particularly intractable problem when it comes to quantitation, especially with current strategies that exhibit limited sensitivity (usually 5-10 ppm). In addition, the existing solutions do not lend themselves to the timelines typically associated with biologics development. FAMS, for example, relies on the production of free fatty acids and typically requires 2-4 weeks before detectable PS degradation occurs and can present a bottleneck for process development or investigations. Accordingly, there is a need in the field for methods capable of highly sensitive quantitation of HCPs in protein samples. In particular, there is a strong need for methods that can be adapted to identify and quantitate the level of HCPs in either a targeted manner (e.g., where the HCPs to be identified and quantitated are predetermined) or a target agnostic manner (e.g., where the HCPs to be identified and quantitated are not predetermined) and which can be modified to achieve a range of sensitivities appropriate for distinct use cases.

### SUMMARY OF THE INVENTION

In a first aspect, the present disclosure is directed to methods for identifying one or more proteins in a sample comprising a protein product, comprising: a) contacting the sample with a protease under conditions sufficient to digest protein present in the sample; b) contacting the sample comprising the digested protein with sodium deoxycholate (SDC) under reducing and heated conditions; c) contacting the sample comprising the digested protein to a chromatographic support to remove undigested protein; d) contacting the chromatographic support with a mobile phase and collecting an eluent; and c) analyzing the eluent using LC-MS/MS to identify one or more proteins in the sample. In certain embodiments, the LC-MS/MS is performed in in data dependent acquisition (DDA) mode.

In certain embodiments, the protein is a host cell protein. In certain embodiments, the host cell protein is an enzyme. In certain embodiments, the enzyme is a hydrolytic enzyme.

In certain embodiments, the protease is trypsin. In certain embodiments, the w/w ratio of protease-to-protein product in the sample is about 1:2000, about 1:800, about 1:400, or about 1:200. In certain embodiments, the digested protein sample is contacted with SDC at about 1% w/v.

In certain embodiments, the chromatographic support is a solid phase extraction support. In certain embodiments, the chromatographic support is a charged surface hybrid support.

In certain embodiments, the method has a limit of detection (LOD) of about 0.1 ppm to about 5 ppm.

In certain embodiments, the protein product is an antibody. In certain embodiments, the sample comprising a protein product is a partially or fully purified sample. In certain embodiments, the partially or fully purified sample is a purification in-process pool sample. In certain embodiments, the in-process pool sample is an ultrafiltration/diafiltration pool sample. In certain embodiments, the in-process pool sample is a drug substance sample. In certain embodiments, the in-process pool sample is a drug product sample

In certain embodiments, load of the protein product in the sample is from about 6 μg to about 300 μg. In certain embodiments, the time of contacting the sample with the protease is from about 2 to about 4 hours. In certain embodiments, the temperature for contacting the sample with a protease is at about 37° C. In certain embodiments, the temperature for contacting the sample comprising the digested protein with SDC is at about 90° C. In certain embodiments, the time of contacting the sample comprising the digested protein with SDC is about 10 minutes.

In another aspect, the present disclosure is directed to methods for determining the ppm level of one or more target proteins in a sample comprising a protein product, comprising: a) contacting the sample with a protease under conditions sufficient to digest the one or more target proteins; b) contacting the sample comprising the one or more digested target proteins with SDC under reducing and heated conditions; c) contacting the sample comprising the one or more digested target proteins to a chromatographic support to remove undigested protein; d) contacting the chromatographic support with a mobile phase and collecting an eluent; and e) analyzing the eluent using LC-MS/MS to identify and quantitate the one or more target protein in the sample, wherein said analysis comprises determining the signals associated with a plurality of standard ppm levels of the one or more target protein and comparing those signals to the signal detected for the one or more target protein in the sample. In certain embodiments, the LC-MS/MS is performed in parallel reaction monitoring (PRM) mode.

In certain embodiments, the one or more target proteins are host cell proteins. In certain embodiments, the host cell protein is an enzyme. In certain embodiments, the enzyme is a hydrolytic enzyme.

In certain embodiments, the protease is trypsin. In certain embodiments, the w/w ratio of protease-to-protein product in the sample is about 1:2000, about 1:800, about 1:400, or about 1:200. In certain embodiments, the one or more digested target protein sample is contacted with SDC at about 0.9% w/v.

In certain embodiments, the chromatographic support is a solid phase extraction support. In certain embodiments, the chromatographic support is a charged surface hybrid support.

In certain embodiments, the method has a Limit of Detection (LOQ) of about 0.01 ppm.

In certain embodiments, the method comprises normalization of data from the LC-MS/MS analysis.

In certain embodiments, the protein product is an antibody. In certain embodiments, the sample comprising a protein product is a partially or fully purified sample. In certain embodiments, the partially or fully purified sample is a purification in-process pools sample. In certain embodiments, the in-process pool sample is a ultrafiltration/diafiltration pool sample. In certain embodiments, the in-process pool sample is a drug substance sample.

In certain embodiments, the load of the protein product in the sample is from about 6 μg to about 300 μg. In certain embodiments, the time of contacting the sample with the protease is from about 2 to about 4 hours. In certain embodiments, the temperature for contacting the sample with a protease is at about 37° C. In certain embodiments, the temperature for contacting the sample comprising the digested protein with SDC is at about 90° C. In certain embodiments, the time of contacting the sample comprising the digested protein with SDC is about 10 minutes.

In another aspect, the present disclosure is directed to methods for identifying one or more proteins in a sample comprising a protein product at a predetermined sensitivity between about 0.1 ppm to about 5 ppm by adjusting the sample load of protein product to achieve desired sensitivity: a) contacting a sample comprising proteins with a protease under conditions sufficient to digest protein present in the sample, b) contacting the sample comprising the digested protein with SDC under reducing and heated conditions; c) contacting the sample comprising the digested protein to a chromatographic support to further remove undigested protein; d) contacting the chromatographic support with a mobile phase and collecting an eluent; e) re-suspending the eluent; and f) analyzing a fraction of the re-suspended eluent using LC-MS/MS to identify one or more proteins in the sample. In certain embodiments, the LC-MS/MS is performed in DDA mode.

In certain embodiments, the fraction of the re-suspended eluent contains about 6 μg, about 30 μg, about 60 μg, about 150 μg, or about 300 μg of protein product.

In certain embodiments, the protein is a host cell protein. In certain embodiments, the host cell protein is an enzyme. In certain embodiments, the enzyme is a hydrolytic enzyme. In certain embodiments, the protease is trypsin. In certain embodiments, the w/w ratio of protease-to-protein product in the sample is about 1:2000, about 1:800, about 1:400, or about 1:200. In certain embodiments, the digested protein sample or the digested target protein sample is contacted with SDC at about 0.9% w/v.

In certain embodiments, the chromatographic support is a solid phase extraction support. In certain embodiments, the chromatographic support is a charged surface hybrid support.

In certain embodiments, the method comprises normalization of data from the liquid chromatography/mass spectrometry analysis.

In certain embodiments, the protein product is an antibody.

In certain embodiments, the sample comprising a protein product is a partially or fully purified sample. In certain embodiments, the partially or fully purified sample is a purification in-process pool sample. In certain embodiments, the in-process pool sample is an antibody ultrafiltration/diafiltration pool sample. In certain embodiments, the in-process pool sample is a drug substance sample. In certain embodiments, the in-process pool sample is a drug product sample.

In certain embodiments, the time of contacting the sample with the protease is from about 2 to about 4 hours. In certain embodiments, the temperature for contacting the sample with a protease is at about 37ºC. In certain embodiments, the temperature for contacting the sample comprising the digested protein with SDC is at about 90° C. In certain embodiments, the time of contacting the sample comprising the digested protein with SDC is about 10 minutes.

### BRIEF DESCRIPTION OF THE DRAWINGS

FIG. 1 depicts the number of HCP peptides and HCP proteins identified using methods described in Example 2, below, in four different mAb-2 UF/DF pool compositions with or without the use of a solid phase extraction (SPE) step. The four different UFDF pool compositions are denoted by A, B, C and D; the use of SPE is contrasted with the absence of SPE in the method (e.g., “A_SPE” and “A”, respectively, in drug product composition A).

FIGS. 2A-2C depict the number of peptides and associated proteins of interest identified using the methods described in Example 3, below, with and without sodium deoxycholate (SDC) addition and with respect to the timing of SDC addition. FIG. 2A depicts results from digest of mAb-1 UF/DF pool spiked with 1 parts per million (ppm) each of five recombinant hydrolases (LPL, PLBL2, SMPD1, PPT1 and LipA), using BEH column, overnight digest, 1:400 trypsin to protein ratio. FIG. 2B depicts the results from digest of mAb-1 UF/DF pool spiked with 0.1 ppm each of eight recombinant hydrolases, using CSH column, 2h digest, 1:2000 trypsin to protein ratio. To assess the reproducibility of the results, the tests were performed in duplicates denoted by Replicate 1 (Repl. 1) and Replicate 2 (Repl. 2). FIG. 2C depicts results from digest of mAb-2 spiked with the 9 identified proteins at 5 ppm and the addition of SDC either during the reducing and heating step (“SDC_reduction1” and “SDC_reduction2”) or to the digest prior to reduction and heating (“SDC_digest1” and “SDC_digest2”).

FIG. 3 depicts the identification of four HCPs of interest in a mAb-1 UF/DF pool composition using the methods described in Example 4, below. As outlined in Example 4, trypsin to protein ratios of 1:400, 1:2000, 1:5000, and 1:10000 were employed at a 25 g/L mAb-1 protein concentration.

FIGS. 4A-4B depicts the identification of eight recombinant hydrolases spiked into a mAb-1 UF/DF pool composition at different trypsin digestion conditions. FIG. 4A depicts the results of eight spiked-hydrolases at approximately 1 ppm (0.58-1.22 ppm) in a mAb-1 at a 30 μg sample load (Note: a 30 μg sample load refers to loading 1.5 μL out of a 100 μL re-suspended digest from a 2 mg mAb-1 drug product composition starting material). FIG. 4B depicts the results of eight spiked-hydrolases at approximately 0.1 ppm (0.058-0.27 ppm) in a mAb-1 at a 400 μg sample load. To assess the reproducibility of the results, the tests were performed in duplicates denoted by Replicate 1 (Repl. 1) and Replicate 2 (Repl. 2).

FIG. 5 depicts the number of spiked-in proteins that were identified at different sample loads. The 8 recombinant hydrolases were spiked in at different levels ranging from 0.1 to 5 ppm. The various sample loads were selected based on the expected sensitivity needs, e.g., a 30 μg starting material load was selected for the 1 ppm spiked sample, as outlined in Example 5. The results indicate that sample load successfully modulates sensitivity at 10 g/L protein concentration of UF/DF pool composition at digestion. To assess the reproducibility of the results, the tests were performed in duplicates denoted by replicate 1 (repl. 1) and replicate 2 (repl. 2), and the LC-MS/MS injections for each test sample were also duplicated (as indicated by A and B).

FIGS. 6A-6B depict the results of Example 6 and demonstrates the impact of trypsin digestion time and protein concentration at digestion on identification of spiked-in recombinant hydrolases through the number of unique peptide counts. Trypin digest times were further fine-tuned to increase the sensitivity and method robustness for identification of eight recombinant hydrolases spiked in at low levels (0.058-0.27 ppm). FIG. 6A depicts the impact of trypsin digestions of 2, 3, and 4 h at 10 g/L protein concentration. FIG. 6B depicts the impact of protein concentration at digestion (10 vs 25 g/L). To assess the reproducibility of the results, the tests were performed in duplicates denoted by replicate 1 (repl. 1) and replicate 2 (repl. 2).

FIG. 7 shows reliable 0.1 ppm sensitivity at 25 g/L protein concentration and 4 h digestion. Unique peptide counts were determined for eight recombinant hydrolases spiked into the protein samples at low levels (0.058-0.27 ppm). The reproducibility/robustness of this method was demonstrated by consistently identifying all hydrolases at ≥0.1 ppm levels using either previously-used or new SPE/nanoLC columns.

FIG. 8 depicts a schematic for a standard addition-based parallel reaction monitoring (PRM) workflow to quantify low levels of targeted HCPs in protein samples.

### DETAILED DESCRIPTION

The present disclosure relates to highly sensitive methods for determining the identity and quantity of one or more proteins in a sample. For example, the subject matter of the present disclosure provides methods for the highly sensitive identification and quantitation of residual host cell proteins in protein samples produced during biologics manufacturing. In certain embodiments, such methods can be adapted to identify and quantitate the level of predetermined proteins (i.e., specific proteins that are targeted for quantitation), e.g., a “target protein” or “targeted protein.” In certain embodiments, such methods can be adapted to be performed in target agnostic manner to identify and quantitate the level of proteins that were not predetermined or selected a priori as targets for quantitation, e.g., “non-targeted proteins”. In certain embodiments, such methods can be modified to achieve a range of sensitivities appropriate for distinct use cases.

For clarity, but not by way of limitation, the detailed description of the presently disclosed subject matter is divided into the following subsections:
1. Definitions;
2. Target Agnostic Methods for Identifying Proteins;
3. Targeted Methods for Quantitating Proteins; and
4. Modulating Sensitivity by Changing Sample Load.

1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

“Culturing” a cell refers to contacting a cell with a cell culture medium under conditions suitable to the survival and/or growth and/or proliferation of the cell.

As used herein, the term “cell,” refers to any suitable prokaryotic or eukaryotic cells. For example, suitable eukaryotic cells include animal cells, e.g., mammalian cells. In certain embodiments, suitable cells are cultured cells. In certain embodiments, suitable cells are host cells, recombinant cells, and recombinant host cells. In certain embodiments, suitable cells are cell lines obtained or derived from mammalian tissues which are able to grow and survive when placed in media containing appropriate nutrients and/or growth factors.

The terms “host cell,” “host cell line” and “host cell culture” are used interchangeably and refer to cells and their progeny into which exogenous nucleic acid can be subsequently introduced to create recombinant cells. These host cells may also have been modified (i.e., engineered) to alter or delete the expression of certain endogenous host cell proteins. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line”. The terms “host cell,” “host cell line” and “host cell culture” may also refer to such recombinant cells and their progeny.

The terms “recombinant cell”, “recombinant cell line” and “recombinant cell culture” are used interchangeably and refer to cells and their progeny into which exogenous nucleic acid has been introduced to enable the expression of recombinant product of interest. The terms “host cell,” “host cell line” and “host cell culture” may also refer to such recombinant cells and their progeny. The recombinant product expressed by such cells may be a recombinant protein, a recombinant viral particle, or a recombinant viral vector.

The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells within the context of the present disclosure can include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CVI line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). In certain embodiments, the mammalian cells include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989).

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

The term “host cell proteins” is used herein in the broadest sense and encompasses the endogenous proteins produced by the cells used in expressing the exogenous nucleic acid (e.g., to produce the polypeptide of interest). Generally, polypeptides of interest are produced by culturing recombinant cells (e.g., mammalian or bacterial cell lines engineered to produce the polypeptide of interest by insertion of a recombinant plasmid containing the gene for that polypeptide). Since the cultured cells used are living organisms, they also produce endogenous proteins in addition to the exogenously introduced polypeptide of interest. Therefore, at the end of the cell culture production process, the cell culture harvest is a complex mixture of proteins made by the cells themselves (host cell proteins or “HCPs”) and the polypeptide of interest (e.g., antibody).

The term “residual host cell proteins” is used to refer to host cell proteins that persist after the cell culture harvest is purified through downstream processing and remain in the purification in-process pools and/or beyond during the biologics manufacturing process (e.g., in the drug substance and/or drug product).

The terms “product”, “protein product” and “recombinant protein product” are used interchangeably and refer to the recombinant product produced by host cells or recombinant cells expressing the exogenous nucleic acid introduced by recombinant technology. The recombinant product expressed by such cells may be a recombinant protein (e.g., antibody), a recombinant viral particle, or a recombinant viral vector.

2. Target Agnostic Methods for Identifying Proteins

In one aspect, the presently disclosed subject matter provides methods for identifying one or more proteins in a sample using a target-agnostic approach, e.g. where the proteins identified are not predetermined. In certain embodiments, the present disclosure provides target agnostic methods for identifying one or more proteins in a sample comprising normalization of data from liquid chromatography/mass spectrometry analysis.

In certain embodiments, the present disclosure provides target agnostic methods for identifying one or more proteins in a sample comprising an antibody. In certain embodiments, the sample comprising an antibody is a partially purified sample. In certain embodiments, the sample comprising an antibody is a purification in-process pools sample such as an antibody ultrafiltration/diafiltration pool sample. In certain embodiments, the sample comprising an antibody is a drug substance or drug product sample.

For example, but not limitation, target agnostic methods disclosed herein for identifying one or more proteins in a sample, e.g. a sample comprising a recombinant protein product, can comprise:
a) contacting a protein-containing sample with a protease under conditions sufficient to digest protein in the sample;
b) contacting the sample comprising the digested protein with SDC under reducing and heated conditions;
c) separating the SDC, undigested protein, and denatured protein from the sample;
d) contacting the sample comprising the digested protein to a chromatographic support to further remove undigested protein;
e) contacting the chromatographic support with a mobile phase and collecting an eluent; and
f) analyzing the eluent using LC-MS/MS, e.g., in data dependent acquisition (DDA) mode, to identify one or more proteins in the sample that are presented at or above the method limit of detection (LOD) level.

Effective removal of undigested proteins from a native digest process prior to LC-MS/MS analysis can improve analysis robustness as undigested protein, if left unremoved, can cause column clogging and reduce the LC column life. The presently disclosed subject matter is based, at least in part, on the discovery that a step of contacting the sample comprising the digested protein to a chromatographic support allows for more effective protein removal from peptides thus allowing for increased LC-MS analysis robustness. Accordingly, in certain embodiments, the methods of the present disclosure comprise a step of contacting the sample comprising the digested protein to a chromatographic support to remove undigested protein. In certain embodiments, the chromatographic support is a charged surface hybrid support. In certain embodiments, the chromatographic support is an SPE support.

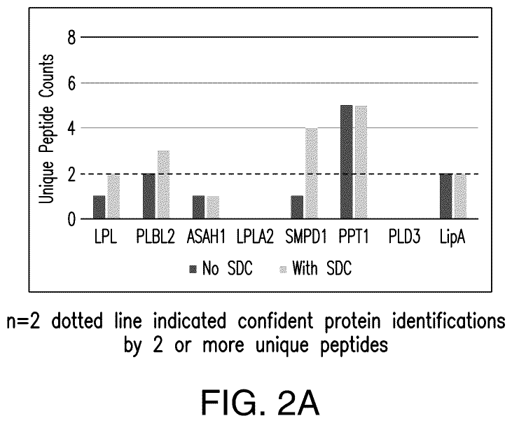

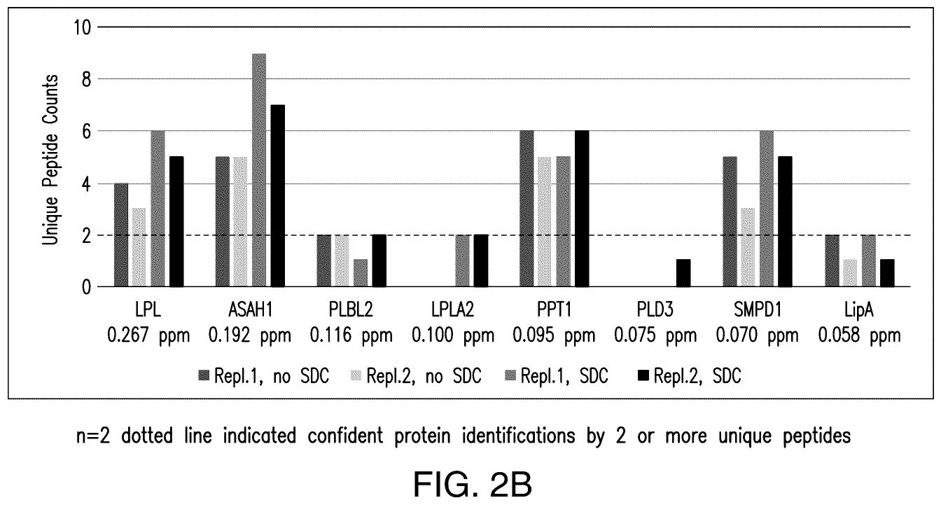

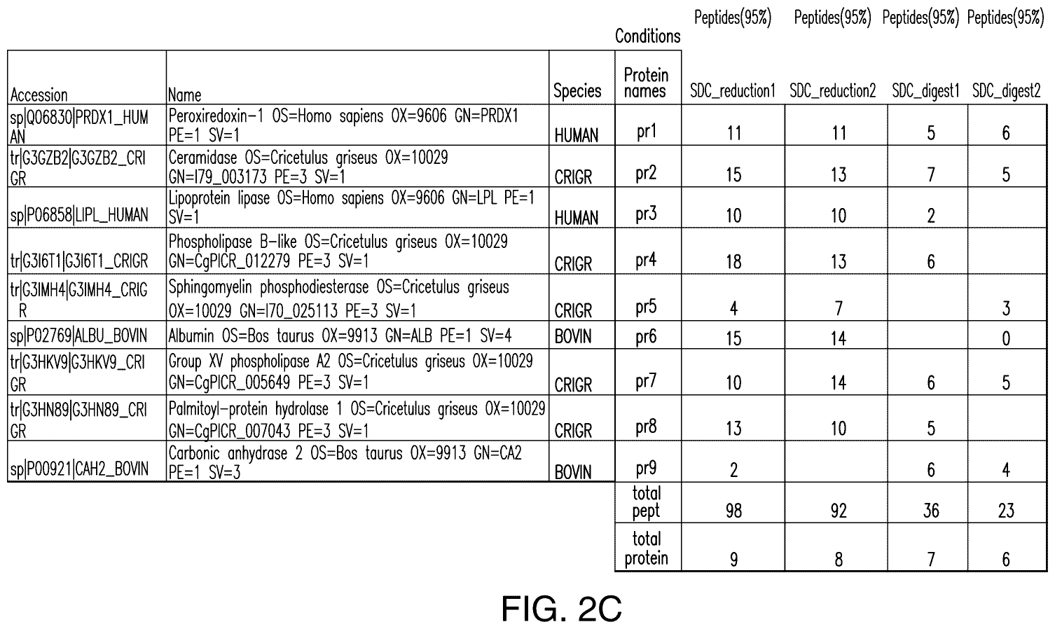

Native digestion strategies can, however, miss proteins, e.g., HCPs, that are associated with a recombinant polypeptide product, such as an mAb product, and hence are present in the original protein composition, e.g., a mAb drug product composition, but which are removed during precipitation. The presently disclosed subject matter is based, at least in part, on the discovery that SDC addition enhances the detection sensitivity of the methods described herein, e.g., for low-abundance HCPs, including those associated with recombinant polypeptides, e.g., mAb products. Without adhering to any theory, the improved peptide, e.g., HCP peptide, identifications with SDC addition may be explained by the ability of SDC to break the strong interactions between proteins, e.g., HCPs, and recombinant polypeptide products, such as mAb products, which can contribute to residual proteins, e.g., HCPs, being present in final drug products. In certain embodiments, the methods of the present disclosure comprise a step of contacting a sample comprising the digested protein product with SDC under reducing and heated conditions. As illustrated in FIG. 2C, methods comprising the contacting of a sample comprising digested protein product with SDC under reducing and heated conditions increased the identification of low-level HCPs as compared to addition of SDC before the reduction. Without being bound by theory, this improvement can be because a native digestion condition is maintained when SDC is added under reduction and heating condition, while addition of SDC before the reduction causes the “native” digestion to become a “denaturing” digestion. A denaturing digestion can increase the digest of mAb and decrease the HCP identification compared to the original native digestion condition. In certain embodiments, the SDC is about 0.9% w/v.

In certain embodiments, the methods of the present disclosure are directed to particularly suitable enzyme-to-protein ratios, e.g., protease-to-protein product ratios, for enzyme digestion, where the protein refers to the concentration of the recombinant protein product, e.g., an mAb product, in the sample being analyzed. For example, in certain embodiments, particular enzyme-to-protein ratios can be used to reduce the overall sample volume for digestion. In certain embodiments, the w/w ratio of enzyme-to-protein in the digest is about 1:200. In certain embodiments, the w/w ratio of enzyme-to-protein in the digest is about 1:400. In certain embodiments, the w/w ratio of enzyme-to-protein in the sample is about 1:800. In certain embodiments, the w/w ratio of enzyme-to-protein in the sample is about 1:4000. In certain embodiments, the w/w ratio of enzyme-to-protein in the sample is about 1:2000. In certain embodiments, the w/w ratio of enzyme-to-protein in the sample is from about 1:200 to about 1:4000. In certain embodiments, the enzyme is a protease. In certain embodiments, the protease is trypsin.

In certain embodiments, the digested protein is diluted to concentrations ranging from 2.5 g/L to 25 g/L of the recombinant protein product in the sample. In certain embodiments, the protein product is diluted to a concentration of 2.5 g/L. In certain embodiments, the protein product is diluted to a concentration of 5 g/L. In certain embodiments, the protein product is diluted to a concentration of 10 g/L. In certain embodiments, the protein product is diluted to a concentration of 25 g/L.

In certain embodiments, the methods of the present disclosure provide that the enzyme-to-protein ratios, e.g., protease-to-protein product ratio, for enzyme digestion is related to the recombinant protein product concentration at digestion. In certain embodiments, different protein product concentrations at digestion with corresponding trypsin to protein ratios, provide increased sensitivity. In certain embodiments, the w/w ratio of enzyme-to-protein in the sample is about 1:200 and the digested protein product is diluted to a concentration of 2.5 g/L. In certain embodiments, the w/w ratio of enzyme-to-protein in the sample is about 1:400 and the protein product is diluted to a concentration of 5 g/L. In certain embodiments, the w/w ratio of enzyme-to-protein in the sample is about 1:800 and the protein product is diluted to a concentration of 10 g/L. In certain embodiments, the w/w ratio of enzyme-to-protein in the sample is about 1:2000 and the protein product is diluted to a concentration of 25 g/L.

In certain embodiments, the presently disclosed subject matter provides target agnostic methods for identifying one or more proteins in a sample comprising contacting a protein-containing sample with a protease under conditions sufficient to digest the protein for about 2 to about 4 hours. In certain embodiments, the protein-containing sample is contacted with a protease under conditions sufficient to digest the protein for about 2 hours. In certain embodiments, the protein-containing sample is contacted with a protease under conditions sufficient to digest the protein for about 3 hours. In certain embodiments, the protein-containing sample is contacted with a protease under conditions sufficient to digest the protein for about 4 hours.

In certain embodiments, the presently disclosed subject matter provides target agnostic methods for identifying one or more proteins wherein the method has a limit of detection (LOD) of about 0.1 parts per million (ppm). In certain embodiments, the method has LOD of about 0.2 ppm. In certain embodiments, the method has a LOD of about 0.5 ppm. In certain embodiments, the method has a LOD of about 1 ppm. In certain embodiments, the method has a LOD of about 5 ppm.

3. Targeted Methods for Quantitating Proteins

In another aspect, the presently disclosed subject matter provides methods for identifying and quantitating one or more targeted protein in a sample. In certain embodiments, quantitation of the one or more targeted proteins comprises determining the ppm level of the one or more targeted proteins.

In certain embodiments, the one or more targeted protein in the sample is a host cell protein such as a host cell enzyme. In certain embodiments, the one or more targeted protein in the sample is a hydrolase.

In certain embodiments, the targeted protein is selected from the group consisting of: N-acylsphingosine amidohydrolase 1 (Acid ceramidase) (ASAH1); Palmitoyl-protein thioesterase 1 (PPT1); Sphingomyelin phosphodiesterase (SMPD1); Lysosomal phospholipase A2 (LPLA2); Lipoprotein lipase (LPL); Lipase A (LIPA); Putative phospholipase B-like 2 (PLBL2); and Phospholipase D family member 3 (PLD3).

In certain embodiments, the targeted protein is selected from the group consisting of: Gelsolin; Lactotransferrin; Serotransferrin (Apotransferrin); Serum Albumin; Catalase; Histidyl-tRNA synthetase; Antithrombin-III; Microtubule-associated protein tau; Creatine kinase M-type; Small ubiquitin-related modifier 1 (SUMO-1); Annexin A 5; NAD(P)H dehydrogenase (quinone) 1; Carbonic anhydrase 2; Carbonic anhydrase 1; Ribosyldihydronicotinamide dehydrogenase; Glutathione S-transferase A1 (GST A1); Glutathione S-transferase P (GST-P); C-reactive protein; Ubiquitin-conjugating enzyme E2 E1 (UbcH6); BH3 Interacting domain death agonist (BID); Peroxiredoxin 1; GTPase HRas (Ras protein); Retinol-binding protein; Ubiquitin-conjugating enzyme E2 C (UbcH10); Peptidyl-prolyl cis-trans isomerase A; Ubiquitin-conjugating enzyme E2 I (UbcH9); Tumor necrosis factor (TNF-alpha); Myoglobin C; Interferon gamma (IFN-gamma); Leptin; Cytochrome b5; Hemoglobin beta chain; Superoxide dismutase (Cu—Zn); Gamma-synuclein; Hemoglobin alpha chain; Fatty acid-binding protein; Lysozyme C; Alpha-lactalbumin; Thioredoxin; Platelet-derived growth factor B chain; Beta-2-microglobulin; Cytochrome c (Apocytochrome c); Ubiquitin; Neddylin (Nedd8); Complement C5 (Complement C5a); Interleukin-8; Insulin-like growth factor II; and Epidermal Growth Factor.

In certain embodiments, the present disclosure provides methods for identifying and quantitating one or more target proteins in a sample comprising an antibody. In certain embodiments, the sample comprising an antibody is a partially purified sample. In certain embodiments, the sample comprising an antibody is a purification in-process pools sample such as an antibody ultrafiltration/diafiltration pool sample. In certain embodiments, the sample comprising an antibody is a drug substance or drug product sample.

In certain embodiments, the present disclosure provides methods for identifying and quantitating one or more target proteins in a sample, e.g. a sample comprising a protein product, comprising:
a) contacting the sample with a protease under conditions sufficient to digest the one or more target proteins present in the sample;
b) contacting the sample comprising the digested target protein with SDC under reducing and heated conditions;
c) separating the SDC, undigested protein, and denatured protein from the sample;
d) contacting the sample comprising the digested target protein to a chromatographic support to further remove undigested protein;
e) contacting the chromatographic support with a mobile phase and collecting an eluent; and
f) analyzing the eluent using LC-MS/MS, e.g., in parallel reaction monitoring (PRM) mode, and comparing the results of such analysis with signals associated with a plurality of standard ppm levels of the one or more target proteins and to identify and quantitate the ppm level of the one or more target proteins in the sample.

In certain embodiments, the methods of the present disclosure comprise the step of contacting the sample comprising the one or more digested target proteins to a chromatographic support to remove undigested protein. In certain embodiments, the chromatographic support is a charged surface hybrid support. In certain embodiments, the chromatographic support is a SPE support.

In certain embodiments, the methods of the present disclosure comprising the step of contacting a sample comprising the one or more digested target protein with SDC under reducing and heated conditions is implemented. In certain embodiments, the SDC is about 0.9% w/v.

In certain embodiments, the methods of the present disclosure are directed to predetermined enzyme-to-protein ratios, e.g., protease-to-protein product ratios, for improved enzyme digestion and overall sensitivity. For example, in certain embodiments, particular enzyme-to-protein ratios can be used to reduce the overall sample volume for digestion. In certain embodiments, the w/w ratio of enzyme-to-protein in the sample is about 1:200. In certain embodiments, the w/w ratio of enzyme-to-protein in the sample is about 1:400. In certain embodiments, the w/w ratio of enzyme-to-protein in the sample is about 1:800. In certain embodiments, the w/w ratio of enzyme-to-protein in the sample is about 1:4000. In certain embodiments, the w/w ratio of enzyme-to-protein in the sample is about 1:2000. In certain embodiments, the enzyme is a protease. In certain embodiments, the protease is trypsin.

In certain embodiments, the methods of the present disclosure provide that the enzyme-to-protein ratios, e.g., protease-to-protein product ratio, for enzyme digestion is related to the recombinant protein product concentration at digestion. In certain embodiments, different protein product concentrations at digestion with corresponding trypsin to protein ratios, provide increased sensitivity. In certain embodiments, the w/w ratio of enzyme-to-protein in the sample is about 1:200 and the protein product is diluted to a concentration of 2.5 g/L. In certain embodiments, the w/w ratio of enzyme-to-protein in the sample is about 1:400 and the protein product is diluted to a concentration of 5 g/L. In certain embodiments, the w/w ratio of enzyme-to-protein in the sample is about 1:800 and the protein product is diluted to a concentration of 10 g/L. In certain embodiments, the w/w ratio of enzyme-to-protein in the sample is about 1:2000 and the protein product is diluted to a concentration of 25 g/L.

In certain embodiments, the presently disclosed subject matter provides methods for identifying and quantitating the ppm level of one or more target proteins in a sample comprising contacting a recombinant protein-containing sample with a protease under conditions sufficient to digest the protein for about 2 to about 4 hours. In certain embodiments, the protein-containing sample is contacted with a protease under conditions sufficient to digest the protein for about 2 hours. In certain embodiments, the protein-containing sample is contacted with a protease under conditions sufficient to digest the protein for about 3 hours. In certain embodiments, the protein-containing sample is contacted with a protease under conditions sufficient to digest the protein for about 4 hours.

In certain embodiments, the presently disclosed subject matter provides methods for identifying and quantitating the ppm level of one or more target proteins wherein the method has a limit of quantitation (LOQ) of about 0.01 ppm. In certain embodiments, analysis of a fraction of the re-suspended eluent can comprise using LC-MS/MS in PRM mode to determine the ppm level of a target protein in a sample can comprise determining the signals associated with a plurality of standard ppm levels of the known target protein and comparing those signals to the signal detected for the known target protein in the sample to thereby achieve a quantitation sensitivity of 0.01 ppm.

4. Modulating Sensitivity by Changing Sample Load

In another aspect, the presently disclosed subject matter provides methods for modulating the sensitivity of the identification and quantitation strategies described herein. In certain embodiments, such modulation of sensitivity can be achieved by selecting the particular amount of protein, e.g., the amount of recombinant protein product, in a sample to be digested in connection with the methods of the present disclosure. For example, but not limitation, the amount of recombinant protein product present in the sample injected for LC-MS/MS analysis can be based on the amount of starting material, e.g., a 30 μg sample load means loading 1.5 μL out of a 100 μL resuspended digest from a 2 mg starting material digest.

In certain embodiments, the present disclosure provides target agnostic methods for identifying one or more proteins in a sample at a predetermined sensitivity between about 0.1 ppm to about 5 ppm. In certain embodiments, the target agnostic methods of identifying one or more proteins in a sample at a predetermined sensitivity of the present disclosure can comprise:
a) contacting a protein-containing sample at a preselected protein product concentration with a protease under conditions sufficient to digest protein;
b) contacting the sample comprising the digested protein with SDC under reducing and heated conditions;
c) separating the SDC, undigested protein, and denatured protein from the sample;
d) contacting the sample comprising the digested protein to a chromatographic support to further remove undigested protein;
e) contacting the chromatographic support with a mobile phase and collecting an eluent; and
f) analyzing the eluent using LC-MS/MS, e.g., in DDA mode, to identify one or more proteins in the sample.

In certain embodiments, the present disclosure provides target agnostic methods for identifying proteins in a sample at a predetermined sensitivity between about 0.1 ppm to about 5 ppm by analyzing a fraction of the re-suspended eluent having a predetermined amount of recombinant protein product via liquid chromatography/mass spectrometry to determine the concentration of the protein in the sample. As noted above, the amount of recombinant protein product present in the sample injected for LC-MS/MS analysis can be determined based on the amount of starting material, e.g., a 30 μg sample load means loading 1.5 μL out of a 100 μL resuspended digest from a 2 mg starting material digest. In certain embodiments, the fraction of the re-suspended eluent contains about 6 μg, about 30 μg, about 60 μg, about 150 μg, or about 300 μg of protein product. In certain embodiments, the fraction of the re-suspended eluent containing about 6 μg of protein product. In certain embodiments, the fraction of the re-suspended eluent containing about 30 μg of protein product. In certain embodiments, the fraction of the re-suspended eluent containing about 60 μg of protein product. In certain embodiments, the fraction of the re-suspended eluent containing about 150 μg of protein product. In certain embodiments, the fraction of the re-suspended eluent containing about 300 μg of protein product.

In certain embodiments, the presently disclosed subject matter provides target agnostic methods for identifying one or more proteins in a sample at a predetermined sensitivity between about 0.1 ppm to about 5 ppm comprising contacting a protein-containing sample with a protease under conditions sufficient to digest the protein for about 2 to about 4 hours. In certain embodiments, the protein-containing sample is contacted with a protease under conditions sufficient to digest the protein for about 2 hours. In certain embodiments, the protein-containing sample is contacted with a protease under conditions sufficient to digest the protein for about 3 hours. In certain embodiments, the protein-containing sample is contacted with a protease under conditions sufficient to digest the protein for about 4 hours.

5. Exemplary Non-limiting Embodiments

A. In certain embodiments, the present disclosure is directed to methods for identifying one or more proteins in a sample comprising a protein product, comprising:
a) contacting the sample with a protease under conditions sufficient to digest protein present in the sample;
b) contacting the sample comprising the digested protein with sodium deoxycholate (SDC) under reducing and heated conditions;
c) contacting the sample comprising the digested protein to a chromatographic support to remove undigested protein;
d) contacting the chromatographic support with a mobile phase and collecting an eluent; and
e) analyzing the eluent using LC-MS/MS to identify one or more proteins in the sample.

A1. In certain embodiments, the present disclosure is directed to the methods of A wherein the LC-MS/MS is performed in in data dependent acquisition (DDA) mode.

A2. In certain embodiments, the present disclosure is directed to the methods of A, wherein the protein is a host cell protein.

A3. In certain embodiments, the present disclosure is directed to the methods of A2, wherein the host cell protein is an enzyme.

A4. In certain embodiments, the present disclosure is directed to the methods of A3 wherein the enzyme is a hydrolytic enzyme.

A5. In certain embodiments, the present disclosure is directed to the methods of A, wherein the protease is trypsin.

A6. In certain embodiments, the present disclosure is directed to the methods of A, wherein the w/w ratio of protease-to-protein product in the sample is about 1:2000, about 1:800, about 1:400, or about 1:200.

A7. In certain embodiments, the present disclosure is directed to the methods of A, wherein the digested protein sample is contacted with SDC at about 1% w/v.

A8. In certain embodiments, the present disclosure is directed to the methods of A, wherein the chromatographic support is a solid phase extraction support.

A9. In certain embodiments, the present disclosure is directed to the methods of A, wherein the chromatographic support is a charged surface hybrid support.

A10. In certain embodiments, the present disclosure is directed to the methods of A, wherein the method has a limit of detection (LOD) of about 0.1 ppm to about 5 ppm.

A11. In certain embodiments, the present disclosure is directed to the methods of A, wherein the protein product is an antibody.

A12. In certain embodiments, the present disclosure is directed to the methods of A, wherein the sample comprising a protein product is a partially or fully purified sample.

A13. In certain embodiments, the present disclosure is directed to the methods of A12, wherein the partially or fully purified sample is a purification in-process pool sample.

A14. In certain embodiments, the present disclosure is directed to the methods of A13, wherein the in-process pool sample is an ultrafiltration/diafiltration pool sample.

A15. In certain embodiments, the present disclosure is directed to the methods of A13, wherein the in-process pool sample is a drug substance sample.

A16. In certain embodiments, the present disclosure is directed to the methods of claim A13, wherein the in-process pool sample is a drug product sample

A17. In certain embodiments, the present disclosure is directed to the methods of A, wherein the load of the protein product in the sample is from about 6 μg to about 300 μg.

A18. In certain embodiments, the present disclosure is directed to the methods of A, wherein the time of contacting the sample with the protease is from about 2 to about 4 hours.

A19. In certain embodiments, the present disclosure is directed to the methods of A, wherein the temperature for contacting the sample with a protease is at about 37° ° C.

A20. In certain embodiments, the present disclosure is directed to the methods of A, wherein the temperature for contacting the sample comprising the digested protein with SDC is at about 90° C.

A21. In certain embodiments, the present disclosure is directed to the methods of A, wherein the time of contacting the sample comprising the digested protein with SDC is about 10 minutes.

B. In certain embodiments, the present disclosure is directed to methods for determining the ppm level of one or more target proteins in a sample comprising a protein product, comprising:
a) contacting the sample with a protease under conditions sufficient to digest the one or more target proteins;
b) contacting the sample comprising the one or more digested target proteins with SDC under reducing and heated conditions;
c) contacting the sample comprising the one or more digested target proteins to a chromatographic support to remove undigested protein;
d) contacting the chromatographic support with a mobile phase and collecting an eluent; and
e) analyzing the eluent using LC-MS/MS to identify and quantitate the one or more target protein in the sample, wherein said analysis comprises determining the signals associated with a plurality of standard ppm levels of the one or more target protein and comparing those signals to the signal detected for the one or more target protein in the sample.

B1. In certain embodiments, the present disclosure is directed to the methods of B, wherein the LC-MS/MS is performed in parallel reaction monitoring (PRM) mode.

B2. In certain embodiments, the present disclosure is directed to the methods of B, wherein the one or more target proteins are host cell proteins.

B3. In certain embodiments, the present disclosure is directed to the methods of B2, wherein the host cell protein is an enzyme.

B4. In certain embodiments, the present disclosure is directed to the methods of B3 wherein the enzyme is a hydrolytic enzyme.

B5. In certain embodiments, the present disclosure is directed to the methods of B, wherein the protease is trypsin.

B6. The method of B, wherein the w/w ratio of protease-to-protein product in the sample is about 1:2000, about 1:800, about 1:400, or about 1:200.

B7. In certain embodiments, the present disclosure is directed to the methods of B, wherein the one or more digested target protein sample is contacted with SDC at about 0.9% w/v.

B8. In certain embodiments, the present disclosure is directed to the methods of B, wherein the chromatographic support is a solid phase extraction support.

B9. In certain embodiments, the present disclosure is directed to the methods of B, wherein the chromatographic support is a charged surface hybrid support.

B10. In certain embodiments, the present disclosure is directed to the methods of B, wherein the method has a LOQ of about 0.01 ppm.

B11. In certain embodiments, the present disclosure is directed to the methods of B, further comprising normalization of data from the LC-MS/MS analysis.

B12. In certain embodiments, the present disclosure is directed to the methods of B, wherein the protein product is an antibody.

B13. In certain embodiments, the present disclosure is directed to the methods of B, wherein the sample comprising a protein product is a partially or fully purified sample.

B14. In certain embodiments, the present disclosure is directed to the methods of B13, wherein the partially or fully purified sample is a purification in-process pool sample.

B15. In certain embodiments, the present disclosure is directed to the methods of B14, wherein the in-process pool sample is a ultrafiltration/diafiltration pool sample.

B16. In certain embodiments, the present disclosure is directed to the methods of B14, wherein the in-process pool sample is a drug substance sample.

B17. In certain embodiments, the present disclosure is directed to the methods of B, wherein the load of the protein product in the sample is from about 6 μg to about 300 μg.

B18. In certain embodiments, the present disclosure is directed to the methods of B, wherein the time of contacting the sample with the protease is from about 2 to about 4 hours.

B19. In certain embodiments, the present disclosure is directed to the methods of B, wherein the temperature for contacting the sample with a protease is at about 37° C.

B20. In certain embodiments, the present disclosure is directed to the methods of B, wherein the temperature for contacting the sample comprising the digested protein with SDC is at about 90° ° C.

B21. In certain embodiments, the present disclosure is directed to the methods of B, wherein the time of contacting the sample comprising the digested protein with SDC is about 10 minutes.

C. In certain embodiments, the present disclosure is directed to methods for identifying one or more proteins in a sample comprising a protein product at a predetermined sensitivity between about 0.1 ppm to about 5 ppm by adjusting the sample load of protein product to achieve desired sensitivity:
a) contacting a sample comprising proteins with a protease under conditions sufficient to digest protein present in the sample,
b) contacting the sample comprising the digested protein with SDC under reducing and heated conditions;
c) contacting the sample comprising the digested protein to a chromatographic support to further remove undigested protein;
d) contacting the chromatographic support with a mobile phase and collecting an eluent;
e) re-suspending the eluent; and
f) analyzing a fraction of the re-suspended eluent using LC-MS/MS to identify one or more proteins in the sample.

C1. In certain embodiments, the present disclosure is directed to methods of C, wherein the LC-MS/MS is performed in DDA mode.

C2. In certain embodiments, the present disclosure is directed to methods of C, therein the fraction of the re-suspended eluent contains about 6 μg, about 30 μg, about 60 μg, about 150 μg, or about 300 μg of protein product.

C3. In certain embodiments, the present disclosure is directed to methods of C, wherein the protein is a host cell protein.

C4. In certain embodiments, the present disclosure is directed to methods of C3, wherein the host cell protein is an enzyme.

C5. In certain embodiments, the present disclosure is directed to methods of C4, wherein the enzyme is a hydrolytic enzyme.

C6. The method of C, wherein the protease is trypsin.

C7. In certain embodiments, the present disclosure is directed to methods of C, wherein the w/w ratio of protease-to-protein product in the sample is about 1:2000, about 1:800, about 1:400, or about 1:200.

C8. In certain embodiments, the present disclosure is directed to methods of C, wherein the digested protein sample or the digested target protein sample is contacted with SDC at about 0.9% w/v.

C9. In certain embodiments, the present disclosure is directed to methods of C, wherein the chromatographic support is a solid phase extraction support.

C10. In certain embodiments, the present disclosure is directed to methods of C, wherein the chromatographic support is a charged surface hybrid support.

C11. In certain embodiments, the present disclosure is directed to methods of C, comprising normalization of data from the liquid chromatography/mass spectrometry analysis.

C12. In certain embodiments, the present disclosure is directed to methods of C, wherein the protein product is an antibody.

C13. In certain embodiments, the present disclosure is directed to methods of C, wherein the sample comprising a protein product is a partially or fully purified sample.

C14. In certain embodiments, the present disclosure is directed to methods of C13, wherein the partially or fully purified sample is a purification in-process pool sample.

C15. In certain embodiments, the present disclosure is directed to methods of C14, wherein the in-process pool sample is an antibody ultrafiltration/diafiltration pool sample.

C16. In certain embodiments, the present disclosure is directed to methods of C14, wherein the in-process pool sample is a drug substance sample.

C17. In certain embodiments, the present disclosure is directed to methods of C14, wherein the in-process pool sample is a drug product sample.

C18. In certain embodiments, the present disclosure is directed to methods of C, wherein the time of contacting the sample with the protease is from about 2 to about 4 hours.

C19. In certain embodiments, the present disclosure is directed to methods of C, wherein the temperature for contacting the sample with a protease is at about 37° C.

C20. In certain embodiments, the present disclosure is directed to methods of C, wherein the temperature for contacting the sample comprising the digested protein with SDC is at about 90° C.

C21. In certain embodiments, the present disclosure is directed to methods of C, wherein the time of contacting the sample comprising the digested protein with SDC is about 10 minutes.

### EXAMPLES

### Materials and Methods

### Materials

The full length mAb-1 (IgG1, full length), mAb-2 (IgG1 Fab), and mAb-3 (IgG1, full length) drug product compositions were produced in-house. Recombinant human lipoprotein lipase (LPL) was purchased from R&D Systems (Minneapolis, MN). All other seven recombinant hydrolases were produced by fusing a C-terminal 6xHis-or dual 6xHis-Flag epitope to each hydrolase as a purification tag. The expression constructs were verified by DNA sequencing and transiently transfected into CHO cells. The recombinant enzymes were harvest 10 days after transfection and purified by Ni-NTA, size exclusion, and anti-His affinity chromatography. The seven recombinant hydrolases were combined with the recombinant LPL to generate the Hydrolase standard (STD). The name, molecular weight (MW), purity of recombinant standards, and level measured in parts per million (ppm), prior to any spiking as described herein, in the mAb-1 drug product composition produced in-house (“Endogenous Level in mAb-1”) of these enzymes are listed in Table 1. Universal Proteomic Standard 1 (UPS-1) was purchased (Sigma-Aldrich) and contains a mixture of 48 recombinant proteins of known equimolar concentration and MW ranging from 6.3 to 82.9 kDa. The humanized IgGIK monoclonal antibody standard RM 8671 was purchased from the National Institute of Standards and Technology (NIST). Recombinant bovine trypsin was from Roche. All reagent stock solutions were prepared in LCMS-grade water.

TABLE 1
The MW, purity of recombinant standards and the endogenous level
of these eight hydrolases in a mAb-1 drug product composition.

| | | | | Purity of | Endogenous |
|---|---|---|---|---|---|
| | | | MW | Recombinant | Level in mAb-1 |
| Name | Symbol | Accession | [kDa] | Standard* (%) | (ppm) |
| Acid ceramidase | ASAH1 | G3GZB2 | 44.7 | 100 | 0.0923 |
| Palmitoyl-protein | Ppt1 | G3HN89 | 34.5 | 95.45 | ND** |
| thioesterase 1 | | | | | |
| Sphingomyelin | Smpd1 | G3IMH4 | 66.2 | 70.41 | ND** |
| phosphodiesterase 1 | | | | | |
| Lysosomal | LPLA2 | G3HKV9 | 47.2 | 99.59 | ND** |
| phospholipase A2 | | | | | |
| Lipoprotein lipase | Lpl | G3H6V7 | 50.5 | 95 | 0.1717 |
| Lipase A | Lipa | G3HQY6 | 45.6 | 58.35 | ND** |
| Putative phospholipase | PLBL2 | G3I6T1 | 65.5 | 100 | 0.0162 |
| B-like 2 | | | | | |
| Phospholipase D | Pld3 | G3HNQ5 | 54.4 | 75.38 | ND** |
| family member 3 | | | | | |

*Recombinant human LPL was purchased from R&D Systems (Minneapolis, MN) and was ~95% pure as per the certificate of analysis provided by the manufacturer. The other 7 recombinant hydrolases are CHO-derived proteins and their purities were assessed by size exclusion chromatography.
**PPT1, SMPD1, LPLA1, LipA, and PLD3 were not detected (ND) in the mAb-1 drug product.

### Equipment

Water bath capable of holding 37° C. (VWR Scientific, Model 1235 or equivalent), dry bath capable of 90° C. (USA Scientific, Model BSH200 or equivalent), and centrifuge capable of high speed and temperature control (Eppendorf, Model 5424 R or equivalent) were used during sample preparation.

### Preparation of HCP Model Samples

A model for low-level HCPs in drug product was prepared by spiking either Hydrolase STD or UPS-1 protein standard solutions into specific mAbs at defined ppm levels, where ppm represents weight of each protein relative to weight of antibody.

### Native Digestion

A 2 mg aliquot of antibody sample was diluted with water and 1 M Tris/HCl buffer (pH 8.0) to the desired protein concentrations ranging from 2.5, 5, 10 and 25 g/L, and digested with bovine trypsin at 1:200, 1:400, 1:800 and 1:2000 enzyme to protein ratio (w/w) respectively at 37° C. for 2 hours, then reduced with 11 mM TCEP (Thermo Scientific, Bondbreaker™ TCEP Solution, 77720) in the presence of 0.9% w/v sodium deoxycholate (SDC) and heated for 10 min in a 90° C. dry bath. The samples are cooled to room temperature and then acidified to pH<2 with 7% formic acid (FA) to quench the digestion and precipitate SDC. The SDC precipitate and undigested and denatured protein were pelleted by a 30 min centrifugation step at 20,000 g and 4° ° C. The supernatant aliquot from the centrifugation step was transferred into a new 1.5 mL low-binding microcentrifuge tube (Eppendorf™ LoBind 022431081, or equivalent) and centrifuged for another 10 min at 20,000 g and 4° C. The final supernatant was cleaned up by an Oasis HLB uElution Plate, eluted with 20 μL of 50% Acetonitrile (ACN), dried down and resuspended in 100 μL of 0.1% formic acid in water. Each processed sample was transferred to 300 μL conical vials (Waters QuanRecovery with MaxPeak, 186009186, or equivalent). Note that the sample injected for LC-MS/MS analysis was based on the amount of starting material, e.g., a 30 μg sample load means loading 1.5 μL out of the 100 μL resuspended digest from a 2 mg starting material.

### Nanoflow RPLC-MS/MS Analysis

LC-MS/MS analysis was performed on an UltiMate 3000 RSLCnano system coupled to Orbitrap Exploris™ 480 Mass Spectrometer (Thermo Fisher Scientific). Peptides were separated with a custom ordered built CSH C18 (130 Å, 1.7 μm, 75 μm×50 cm) column from CoAnn Technologies (Richland, WA). Mobile phases were 0.1% FA in water (Mobile Phase A, PH˜ 2.7) and 0.1% FA in ACN (Mobile Phase B). All separation gradients were run with a 250 nL/min flow rate and 60° C. column temperature, and samples were maintained in the autosampler at 6° C. Digests were loaded at 5 μl/min with 0% B on to an online Waters NanoEase M/Z Symmetry C18 column, 100 Å, 5 μm, 180 μm×2 cm Trap Column and washed for 10 min with 0% mobile phase B, before separation by a 172-min LC gradient (250 nL/min): from 1 to 5% B in 1 min, 5 to 8% B in 6 min, 8 to 15% B in 42 min, 15 to 24% B in 81 min, 24 to 32% B in 31 min, 32 to 40% B in 5 min, 40 to 50% in 5 min, 50% to 1% B in 1 min. Each LC gradient is also followed by a 1% B nano-column equilibration for 33 min. The mass spectrometer was operated in data-dependent mode for the 12 most intense ions. Peptides were subjected to higher-energy collisional dissociation (HCD) fragmentation with the normalized collision energy (NCE) at 30% for each full MS scan at 60,000 resolution, 300% of normalized automatic gain control (AGC) target, 100 ms maximum injection time, m/z 300-1500, and MS/MS events at 15,000 resolution at 100% of normalized AGC target, 100 ms maximum injection time.

### Data Analysis for HCP Identification

For HCP identification, LC-MS/MS data files were searched against a database containing sequences of relevant biotherapeutic products, plus those of all spiked protein standards, concatenated to the CHO Canonical and Isoform database from Uniprot.org (35,256 entries) and other human contaminants, using Thermo Proteome Discoverer software (version 1.4) with SEQUEST search. The database containing the NIST mAb sequence concatenated to the mouse (Mus musculus) Swiss-Prot database from Uniprot.org (February 2021 version; 17,068 entries) was used for HCP analysis in NIST mAb. A protein is required to have two unique peptides (at 5% false discovery rate for peptides, evaluated with a decoy database search) for positive identification.

### Example 1: Development of a Highly Sensitive and Robust Native-Digest Based 1D LC-MS/MS Workflow

A typical native digestion-based workflow includes four key steps: 1) trypsin digestion under nondenaturing conditions, 2) reduction and heating, 3) removal of the undigested antibody (mainly Fab and Fc domain fragments) by centrifugation, and 4) removing the supernatant for LC-MS/MS analysis.

To allow for multiple studies under this study, unless otherwise noted, eight purified recombinant hydrolase proteins spiked into drug product mAb-1 were used to assess parameters for trypsin digestion conditions and other sample preparation steps that affect sensitive and robust HCP identification and quantitation. Since these proteins have different levels of purity and some of them are present at very low endogenous levels prior to being spiked into the drug product (Table 1 “Endogenous Level”), their final spike levels after spiking are different even when they are spiked at the same target level: only five of the eight hydrolases are at or above the targeted spike level. For example, in the 0.1 ppm spiked sample, the actual levels of eight hydrolases ranged from 0.058 to 0.267 ppm, and only 5 of the 8 protein are at or above the targeted 0.1 ppm spike level.

### Example 2: Offline SPE Cleanup for Method Robustness

Effective removal of undigested mAb from the native digest is important prior to LC-MS/MS analysis to ensure analysis robustness. From LC-MS/MS analysis, it was observed that even after two repeated centrifugations at 20,000 g, the undigested mAb was still not completely removed and caused either column clogging or shortened column life, as indicated by built-up column pressure or greatly reduced column performance. To increase analysis robustness and further remove undigested mAb left in the sample, a SPE step was implemented with improved elution conditions. The eluted samples under both 80% and 50% ACN elution conditions (n=2) were clear solutions. However, after drying the sample to the proper volume for ACN removal, a small amount of precipitate was observed for sample with 80% ACN elution; the 50% ACN eluted sample remained clear. Some denatured mAb might have remained in the solution after centrifuge and was eluted from SPE with 80% ACN and then precipitated after the ACN was mostly removed by SpeedVac. Therefore, the elution condition with a lower level of ACN (50%) was selected for SPE cleanup. This 50% ACN level was sufficient to recover desired digested HCP peptides as most peptides usually elute from RP column at <25% ACN, and still can the relatively large and hydrophobic undigested mAb can still be retained on the SPE.

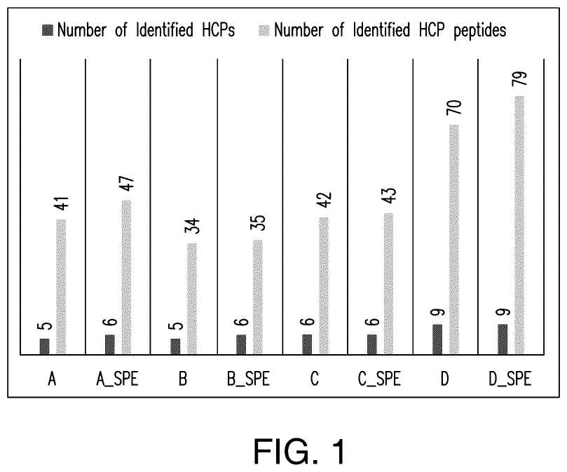

As shown in FIG. 1, overall, the method with additional SPE cleanup identified comparable number of proteins and slightly more peptides to the one without SPE consistently from four different UFDF pool samples in mAb-2. Most importantly, the additional SPE clean-up afforded a long column life and more consistent column pressures after multiple sample loads, which are critical features in a robust assay.

### Example 3: SDC Addition for Enhanced Detection Sensitivity of Low-Level HCPs

For the full length mAb-1 sample spiked with seven HCPs at 1 ppm, as shown in FIG. 2A, the addition of SDC (0.9%) at reduction/heating step increased the total number of identified peptides (16 vs 11) and proteins (5 vs 3) for the five spiked HCPs (LPLA2, SMPD1, PPT1, PLD3 and LipA) at 1 ppm and three endogenous HCPs (LPL, PLBL2 and ASAH1) in mAb-1. Therefore, SDC addition was implemented to the native digestion protocol. SDC can be removed at the same step for protein precipitation. The advantage of the SDC addition method was further evaluated for detecting low level residual HCPs at 0.1 ppm. SDC addition increased the peptide identifications for the eight-spiked hydrolases at level from 0.058 to 0.267 ppm (FIG. 2B), and identified (n=2) an additional protein, LPLA2, which has been reported to cause PS degradation at <0.1 ppm and therefore requires a high sensitivity method to detect and quantify. Table 2 shows additional experiments demonstrating that the SDC addition approach increased overall peptide identifications for UPS-1 proteins spiked at 0.1-1.3 ppm levels in the mAb-3, which adds method robustness for low level protein identifications.

TABLE 2
Number of peptides and proteins identified for UPS-1
proteins spiked into mAb-3 at 0.1-1.3 ppm.

| Sample | Proteins | Peptides |
|---|---|---|
| without SDC_rep1 | 39 | 292 |
| without SDC_rep2 | 41 | 296 |
| with SDC_rep1 | 41 | 355 |
| with SDC_rep2 | 40 | 309 |

The improved HCP/peptide identifications with SDC addition can likely be explained by the ability of SDC to break the strong interactions between HCP and mAb, which have greatly contributed to the residual HCPs presented in the final drug product. Previous studies have highlighted how native digestion could miss HCPs that associate with mAb and hence are removed during precipitation, which was further confirmed by the present results with and without SDC (FIG. 2B). In addition, the timing of SDC addition can impact the identification of low-level HCPs. For example, experiments were conducted with mAb-2 spiked with 9 proteins at 5 ppm and the addition of SDC either during the reducing and heating step (“SDC_reduction1” and “SDC_reduction2”) or to the digest prior to reduction and heating (“SDC_digest1” and “SDC_digest2”). As illustrated in FIG. 2C, Addition of SDC under reducing and heated conditions increased the identification of low-level HCPs as compared to addition of SDC prior to the reduction. Our discoveries show that SDC plays a surprisingly effective role in improving the recovery of low-level HCPs under native digestion condition.

### Example 4: Protein Concentration and Trypsin Ratio

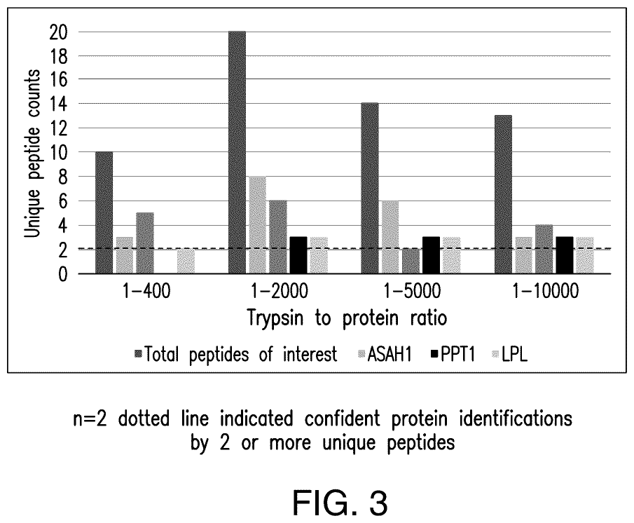

Trypsin-to-protein ratio and digestion time are can be evaluated to enhance trypsin digestion. A previous study showed the advantage of the 2 h digestion time compared to overnight at 1:400 trypsin to protein ratio used in the original native digestion protocol at 5 g/L protein concentration. When analytical flow LC-MS analysis was first used, a large amount of starting material (˜20 mg) was required to detect low level (0.1 ppm and above) HCPs. Therefore, to reduce the sample volume for digestion and sample load, the trypsin to protein ratio was tested at a higher protein concentration of 25 g/L, instead of 5 g/L with 2 h digestion time. FIG. 3 shows the number of peptides for the target proteins (hydrolases) identified in mAb-1 with different trypsin-to-protein ratios. Most peptides from the four target proteins were obtained with 1:2000 trypsin to protein ratio for the 25 g/L protein concentration at trypsin digestion. Experiment with a 0.1 to 1.3 ppm UPS-1 spiked mAb-1 also confirmed that the 1:2000 trypsin to protein ratio identified one more (35 vs 34) UPS-1 protein and 60 more (266 vs 206) UPS-1 peptides than the 1:400 ratio at 25 g/L protein concentration.

A trypsin-to-protein ratio (1:2000 ratio for 25 g/L protein concentration and 1:400 ratio for 5 g/L protein concentration) with 2 h digestion seems to be related to the protein concentration at digestion. To test the hypothesis, and also to establish a predictable digestion conditions for representative drug substance or drug product samples with a broad range of concentrations, the combinations of different protein concentrations (2.5, 5, 10 and 25 g/L) at digestion with their corresponding trypsin-to-protein ratios (1:200, 1:400, 1:800 and 1:2000 ratio), and their impact on the HCP identifications were evaluated.

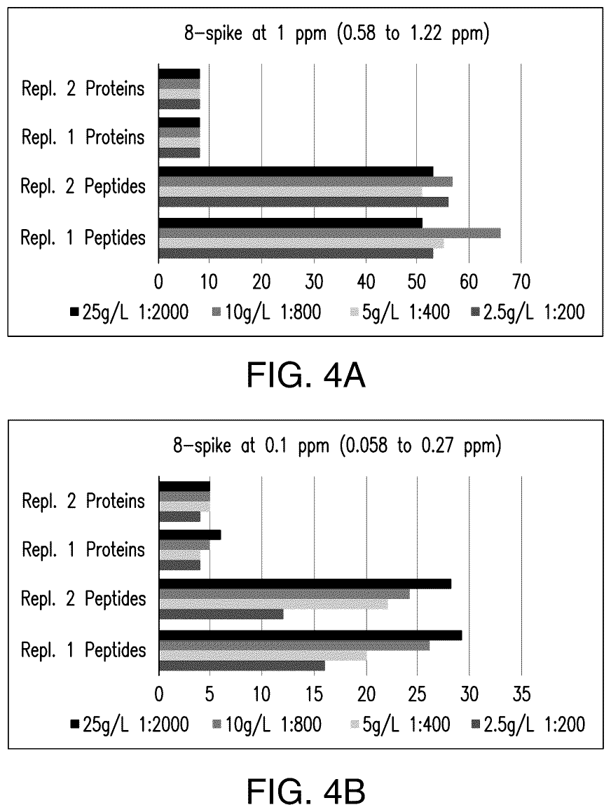

Data were generated for samples spiked with ˜1 ppm of eight recombinant hydrolases (actual levels ranged from 0.584 to 1.122 ppm) under each digestion conditions (n=2). As shown in FIG. 4A, all digestion conditions were able to identify all these eight spiked proteins at 30 μg sample load. This confirms that similar analysis sensitivity can be achieved when protein to trypsin ratio increases inverse linearly to the protein concentration at digestion. Therefore, to achieve 1 ppm level sensitivity, based on the original protein concentration of samples, different digestion strategy can be employed, using the tested combinations of protein concentrations at digestion and their corresponding trypsin to protein ratio.

The same experiment was repeated for the eight recombinant hydrolases spiked in at ˜0.1 ppm (actual levels ranged from 0.058 to 0.267 ppm, with five proteins at or near 0.1 ppm and above) with 400 μg sample load. As shown in FIG. 4B, all conditions identified four to six out of eight hydrolases. The higher (10 and 25 g/L) protein concentrations at digestion identified more peptides in both process replicates compared to the 2.5 and 5 g/L conditions. Taking into consideration the results from both experiment results, the 10 g/L protein concentration was selected with 1:800 trypsin to protein ratio at digestion to test modulating sensitivity with sample load.

### Example 5: Sample Load Modulated Method Sensitivity

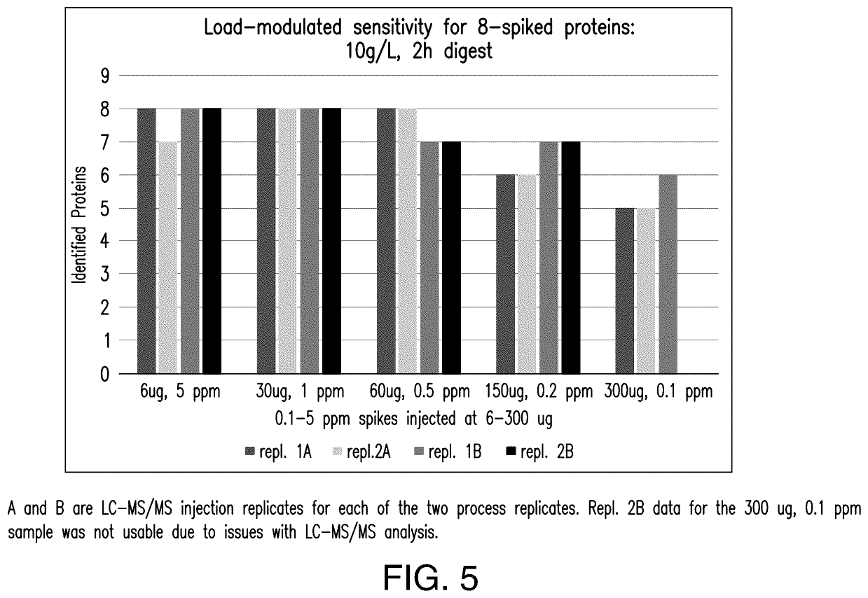

Since the 30 μg starting material analysis showed 1 ppm sensitivity (FIG. 4A), the hypothesis that sample load within acceptable range for LC-MS/MS analysis can modulate sensitivity proportionally was tested, i.e., from the same digest, the 6 μg load is expected to show 5 ppm sensitivity, and 300 μg load is expected to show 0.1 ppm sensitivity. Samples with different spike levels ranging from 0.1 to 5 ppm were digested and different sample loads were selected by on the expected sensitivity needs. e.g., 30 μg starting material load was selected for the 1 ppm spiked sample. The 50 cm-long nanoflow CSH column was used to allow higher sample load to increase sensitivity while maintaining good peak resolution, based on previous reports with analytical flow CSH columns. As shown in FIG. 5, at sample load conditions (6 to 60 μg starting material) for 0.5 to 5 ppm sensitivity, all eight proteins were identified at least once from each of the two process replicates; all five proteins at and above the target sensitivity level were identified in both process replicates. The predicted 0.1 and 0.2 ppm sensitivity were also demonstrated at the tested sample load, by consistently identifying four out of five hydrolases at and above the target sensitivity levels, and two out three below the targeted levels out of the total eight hydrolases.

With higher sensitivity of LC-MS analysis, more residual HCPs will be identified. The level of each HCP also needs to be determined prior to assessing the associated safety risk. The modulated sensitivity provides a tool to rapidly estimate the levels of identified HCPs before more targeted quantitation efforts are made. For example, if X, Y and Z number of proteins are identified at 6, 30 and 300 μg load analyses, it suggests that the corresponding proteins are most likely presented at ≥ 5, 1 and 0.1 ppm respectively. For the samples with X and Y number of proteins identified, the different (i.e., non-overlapping) proteins are potentially present at between 1 and 5 ppm; similarly, for the samples with Y and Z number of proteins identified, the different proteins are potentially present at between 0.1 to 1 ppm. This information will help estimate the levels of each protein identified and support the decision on the need for further targeted quantitation to obtain an accurate HCP measurement.

### Example 6: Improved Sensitivity and Robustness for 0.1 ppm Level

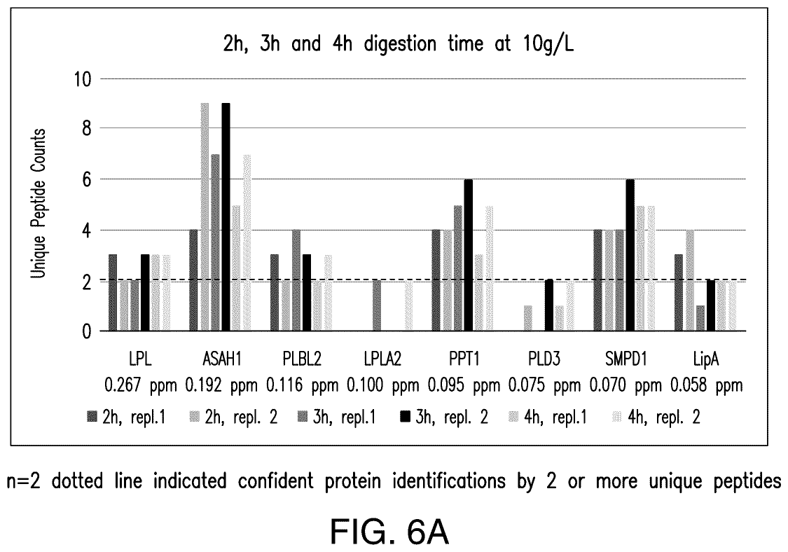

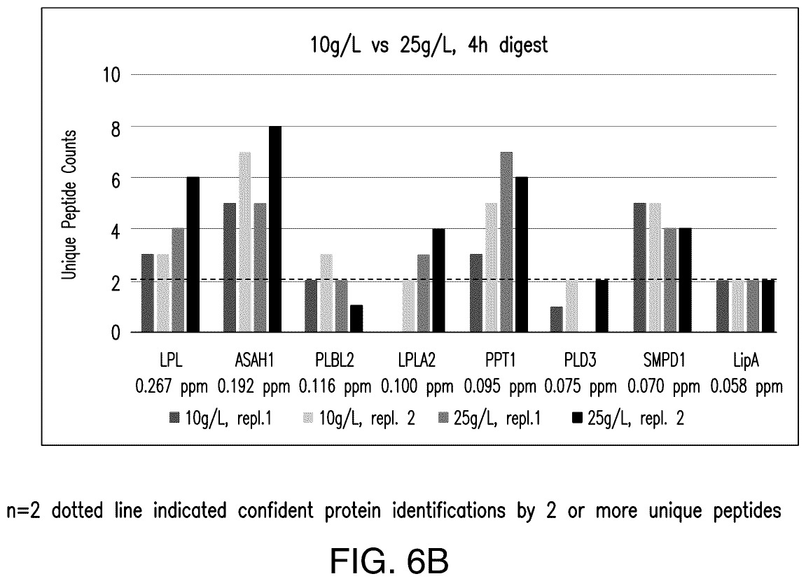

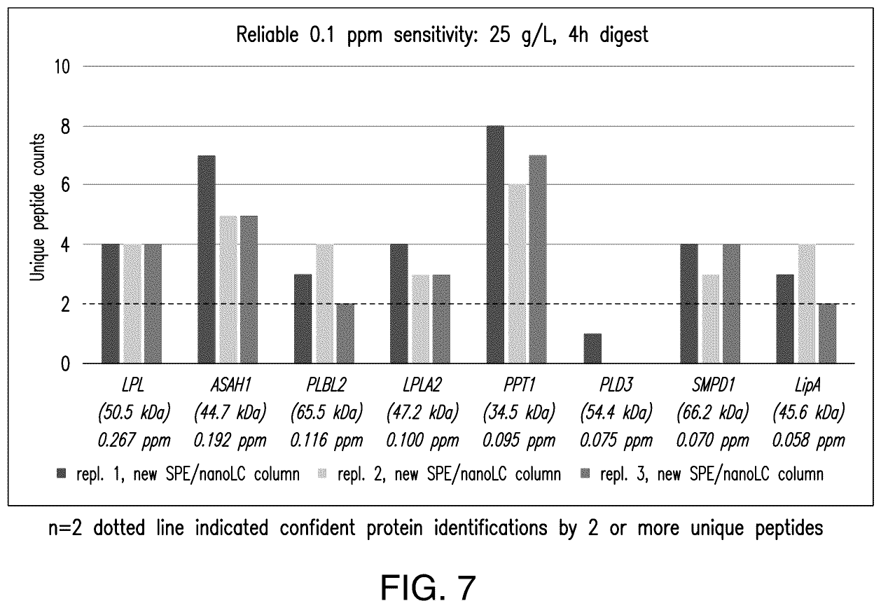

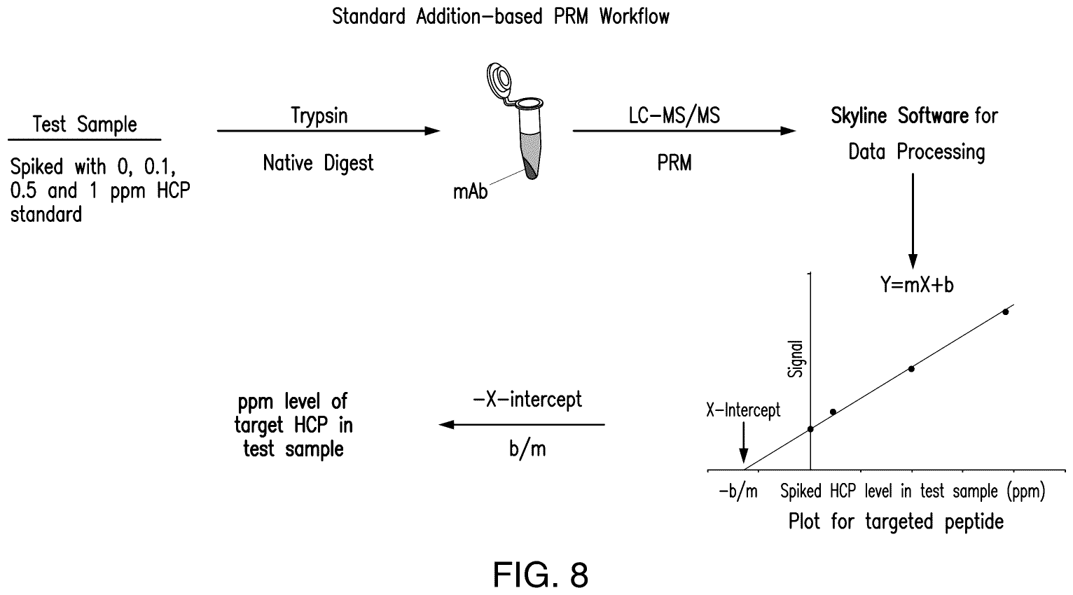

To further fine-tune the sensitivity and increase method robustness for low-level (0.1 ppm), trypsin digest times at 10 g/L were then evaluated. The 4 h digestion time identified the lowest level spiked protein LipA (0.058 ppm) in both process replicates and all the eight proteins at least once, and therefore was considered the best condition (FIG. 6A). Since 25 g/L protein concentration at digestion consistently showed better detection of LPLA2, the 4 h trypsin digest for 25 and 10 g/L protein concentrations was further compared. FIG. 6B shows 25 g/L 4 h condition identified all five spiked proteins at >=0.1 ppm, and two out of three spiked proteins at <0.1 ppm under all tested conditions, while 10 g/L failed to identify LPLA2 at 0.1 ppm and had overall less peptide and proteins identified. FIG. 7 demonstrates consistent 0.058 ppm sensitivity for LipA and robustness for identifying other hydrolases at 0.1 ppm or above (n=3). These results with this method represent a significant improvement in sensitivity over current state-of-the-art methods: >100-fold enhancement over most of the previously reported method sensitivity (10 ppm) and ˜10-fold sensitivity enhancement compared to the recent reports with native digest methods.

### Example 7: Identification of UPS-1 Proteins Spiked in mAb-3

To further confirm the effectiveness of modulating the sample load to achieve the desired sensitivity of protein identification, the mAb-3 sample spiked with known levels of the 48 UPS-1 proteins to simulate trace level HCPs were analyzed. The mAb-3 sample with UPS-1 proteins spiked between 0.10 to 1.34 ppm levels was used to confirm the 0.1 ppm sensitivity. Table 3 shows that 41(85.4%) or more UPS-1 proteins were consistently identified at or above 0.1 ppm (˜4.8 femtomole each) at 300 μg sample load, demonstrating the expected sensitivity (n=2) across a broad MW range of proteins (6.3 to 82.9 kDa MW). Similarly, another mAb-3 sample containing UPS-1 proteins at 0.16 to 2.21 ppm range was used to confirm the 1 ppm sensitivity using digest from 30 μg of starting material. All 16 UPS-1 proteins between 0.59 and 2.21 ppm levels were identified (n=2) (Table 4), confirming the desired 1 ppm sensitivity.

TABLE 3
Detection of 48 UPS-1 proteins spiked in mAb-3 at 300 μg sample load at 0.1 ppm and above.

| UPS-1 | | | | Peptide Counts | | |
|---|---|---|---|---|---|---|
| Index | Accession | MW(Da) | ppm | Repl. 1 | Repl 2 | Description |
| 1 | P06396 | 82959 | 1.34 | 14 | 12 | Gelsolin |
| 2 | P02788 | 76165 | 1.23 | 15 | 13 | Lactotransferrin |
| 3 | P02787 | 75181 | 1.22 | 11 | 12 | Serotransferrin [Apotransferrin] |
| 4 | P02768 | 66357 | 1.07 | 6 | 5 | Serum Albumin |
| 5 | P04040 | 59625 | 0.96 | 16 | 18 | Catalase |
| 6 | P12081 | 58233 | 0.94 | 18 | 17 | Histidyl-tRNA synthetase |
| 7 | P01008 | 49039 | 0.79 | 12 | 13 | Antithrombin-III |
| 8 | P10636-8 | 45719 | 0.74 | 17 | 18 | Microtubule-associated protein tau |
| 9 | P06732 | 43101 | 0.70 | 9 | 11 | Creatine kinase M-type |
| 10 | P63165 | 38815 | 0.63 | 11 | 11 | Small ubiquitin-related modifier 1 [SUMO-1] |
| 11 | P08758 | 35806 | 0.58 | 10 | 8 | Annexin A 5 |
| 12 | P15559 | 30736 | 0.50 | 5 | 5 | NAD(P)H dehydrogenase [quinone] 1 |
| 13 | P00918 | 29115 | 0.47 | 10 | 13 | Carbonic anhydrase 2 |
| 14 | P00915 | 28738 | 0.46 | 6 | 8 | Carbonic anhydrase 1 |
| 15 | P16083 | 25821 | 0.42 | 6 | 7 | Ribosyldihydronicotinamide dehydrogenase |
| 16 | P08263 | 25500 | 0.41 | 6 | 6 | Glutathione S-transferase A1 [GST A1] |
| 17 | P09211 | 23225 | 0.38 | 4 | 2 | Glutathione S-transferase P [GST-P] |
| 18 | P02741 | 23047 | 0.37 | 2 | 2 | C-reactive protein |
| 19 | P51965 | 22227 | 0.36 | 0 | 0 | Ubiquitin-conjugating enzyme E2 E1 [UbcH6] |
| 20 | P55957 | 21995 | 0.36 | 6 | 5 | BH3 Interacting domain death agonist [BID] |
| 21 | Q06830 | 21979 | 0.36 | 2 | 1 | Peroxiredoxin 1 |
| 22 | P01112 | 21298 | 0.34 | 5 | 5 | GTPase HRas [Ras protein] |
| 23 | P02753 | 21071 | 0.34 | 2 | 1 | Retinol-binding protein |
| 24 | O00762 | 20475 | 0.33 | 6 | 6 | Ubiquitin-conjugating enzyme E2 C [UbcH10] |
| 25 | P62937 | 20176 | 0.33 | 5 | 3 | Peptidyl-prolyl cis-trans isomerase A |
| 26 | P63279 | 18007 | 0.29 | 5 | 4 | Ubiquitin-conjugating enzyme E2 I [UbcH9] |
| 27 | P01375 | 17353 | 0.28 | 4 | 5 | Tumor necrosis factor [TNF-alpha] |
| 28 | P02144 | 17053 | 0.28 | 6 | 5 | Myoglobin C |
| 29 | P01579 | 16879 | 0.27 | 5 | 4 | Interferon gamma (IFN-gamma) |
| 30 | P41159 | 16158 | 0.26 | 3 | 3 | Leptin |
| 31 | P00167 | 16022 | 0.26 | 5 | 6 | Cytochrome b5 |
| 32 | P68871 | 15867 | 0.26 | 6 | 5 | Hemoglobin beta chain |
| 33 | P00441 | 15805 | 0.26 | 1 | 1 | Superoxide dismutase [Cu—Zn] |
| 34 | O76070 | 15363 | 0.25 | 9 | 10 | Gamma-synuclein |
| 35 | P69905 | 15126 | 0.24 | 5 | 4 | Hemoglobin alpha chain |
| 36 | P05413 | 14727 | 0.24 | 8 | 6 | Fatty acid-binding protein |
| 37 | P61626 | 14701 | 0.24 | 4 | 4 | Lysozyme C |
| 38 | P00709 | 14078 | 0.23 | 2 | 2 | Alpha-lactalbumin |
| 39 | P10599 | 12429 | 0.20 | 0 | 0 | Thioredoxin |
| 40 | P01127 | 12294 | 0.20 | 0 | 0 | Platelet-derived growth factor B chain |
| 41 | P61769 | 11731 | 0.19 | 6 | 5 | Beta-2-microglobulin |
| 42 | P99999 | 11618 | 0.19 | 4 | 4 | Cytochrome c [Apocytochrome c] |
| 43 | P62988 | 10597 | 0.17 | 3 | 2 | Ubiquitin |
| 44 | Q15843 | 9072 | 0.15 | 3 | 3 | Neddylin [Nedd8] |
| 45 | P01031 | 8536 | 0.14 | 2 | 2 | Complement C5 [Complement C5a] |
| 46 | P10145 | 8386 | 0.14 | 2 | 2 | Interleukin-8 |
| 47 | P01344 | 7475 | 0.12 | 2 | 2 | Insulin-like growth factor II |
| 48 | P01133 | 6353 | 0.10 | 0 | 0 | Epidermal Growth Factor |

TABLE 4
Detection of 48 UPS-1 proteins spiked in mAb-3 at 30 μg load at 1 ppm and above.

| UPS-1 | | | | Peptide Counts | | |
|---|---|---|---|---|---|---|
| Index | Accession | MW(Da) | ppm | Repl. 1 | Repl. 2 | Description |
| 1 | P06396 | 82959 | 2.11 | 10 | 7 | Gelsolin |
| 2 | P02788 | 76165 | 1.93 | 8 | 8 | Lactotransferrin |
| 3 | P02787 | 75181 | 1.91 | 7 | 7 | Serotransferrin [Apotransferrin] |
| 4 | P02768 | 66357 | 1.69 | 5 | 4 | Serum Albumin |
| 5 | P04040 | 59625 | 1.51 | 14 | 12 | Catalase |
| 6 | P12081 | 58233 | 1.48 | 13 | 15 | Histidyl-tRNA synthetase |
| 7 | P01008 | 49039 | 1.25 | 13 | 13 | Antithrombin-III |
| 8 | P10636-8 | 45719 | 1.16 | 15 | 13 | Microtubule-associated protein tau |
| 9 | P06732 | 43101 | 1.09 | 9 | 7 | Creatine kinase M-type |
| 10 | P63165 | 38815 | 0.99 | 5 | 6 | Small ubiquitin-related modifier 1 |
| | | | | | | [SUMO-1] |
| 11 | P08758 | 35806 | 0.91 | 8 | 8 | Annexin A 5 |
| 12 | P15559 | 30736 | 0.78 | 6 | 8 | NAD(P)H dehydrogenase [quinone] 1 |
| 13 | P00918 | 29115 | 0.74 | 10 | 9 | Carbonic anhydrase 2 |
| 14 | P00915 | 28738 | 0.73 | 7 | 7 | Carbonic anhydrase 1 |
| 15 | P16083 | 25821 | 0.66 | 7 | 6 | Ribosyldihydronicotinamide |
| | | | | | | dehydrogenase |
| 16 | P08263 | 25500 | 0.65 | 9 | 10 | Glutathione S-transferase A1 [GST A1] |
| 17 | P09211 | 23225 | 0.59 | 1 | 0 | Glutathione S-transferase P [GST-P] |
| 18 | P02741 | 23047 | 0.59 | 0 | 1 | C-reactive protein |
| 19 | P51965 | 22227 | 0.56 | 0 | 0 | Ubiquitin-conjugating enzyme E2 E1 |
| | | | | | | [UbcH6] |
| 20 | P55957 | 21995 | 0.56 | 3 | 4 | BH3 Interacting domain death agonist |
| | | | | | | [BID] |
| 21 | Q06830 | 21979 | 0.56 | 3 | 1 | Peroxiredoxin 1 |
| 22 | P01112 | 21298 | 0.54 | 4 | 2 | GTPase HRas [Ras protein] |
| 23 | P02753 | 21071 | 0.54 | 1 | 1 | Retinol-binding protein |
| 24 | O00762 | 20475 | 0.52 | 6 | 6 | Ubiquitin-conjugating enzyme E2 C |
| | | | | | | [UbcH10] |
| 25 | P62937 | 20176 | 0.51 | 3 | 4 | Peptidyl-prolyl cis-trans isomerase A |
| 26 | P63279 | 18007 | 0.46 | 3 | 2 | Ubiquitin-conjugating enzyme E2 I |
| | | | | | | [UbcH9] |
| 27 | P01375 | 17353 | 0.44 | 4 | 4 | Tumor necrosis factor [TNF-alpha] |
| 28 | P02144 | 17053 | 0.43 | 5 | 4 | Myoglobin C |
| 29 | P01579 | 16879 | 0.43 | 5 | 5 | Interferon gamma (IFN-gamma) |
| 30 | P41159 | 16158 | 0.41 | 3 | 3 | Leptin |
| 31 | P00167 | 16022 | 0.41 | 5 | 5 | Cytochrome b5 |
| 32 | P68871 | 15867 | 0.40 | 5 | 4 | Hemoglobin beta chain |
| 33 | P00441 | 15805 | 0.40 | 1 | 2 | Superoxide dismutase [Cu—Zn] |
| 34 | O76070 | 15363 | 0.39 | 8 | 8 | Gamma-synuclein |
| 35 | P69905 | 15126 | 0.38 | 5 | 5 | Hemoglobin alpha chain |
| 36 | P05413 | 14727 | 0.37 | 8 | 9 | Fatty acid-binding protein |
| 37 | P61626 | 14701 | 0.37 | 5 | 3 | Lysozyme C |
| 38 | P00709 | 14078 | 0.36 | 2 | 2 | Alpha-lactalbumin |
| 39 | P10599 | 12429 | 0.32 | 0 | 0 | Thioredoxin |
| 40 | P01127 | 12294 | 0.31 | 0 | 0 | Platelet-derived growth factor B chain |
| 41 | P61769 | 11731 | 0.30 | 4 | 4 | Beta-2-microglobulin |
| 42 | P99999 | 11618 | 0.30 | 2 | 4 | Cytochrome c [Apocytochrome c] |
| 43 | P62988 | 10597 | 0.27 | 1 | 2 | Ubiquitin |
| 44 | Q15843 | 9072 | 0.23 | 2 | 2 | Neddylin [Nedd8] |
| 45 | P01031 | 8536 | 0.22 | 1 | 1 | Complement C5 [Complement C5a] |
| 46 | P10145 | 8386 | 0.21 | 2 | 1 | Interleukin-8 |
| 47 | P01344 | 7475 | 0.19 | 1 | 1 | Insulin-like growth factor II |
| 48 | P01133 | 6353 | 0.16 | 0 | 0 | Epidermal Growth Factor |

### Example 8: HCPs Identified in RM8671 NIST mAb

To further demonstrate the sensitivity and robustness of the target agnostic methods for identifying proteins, e.g., residual HCPs, and to enable comparison to recent reports, analyses were performed for RM8671 NIST antibody. This method is considered a target agnostic approach because it does not rely on a priori knowledge of the presence of these specific HCPs in these samples. An average of 599.5 proteins and 3589.5 unique peptides (30 μg load analyses) and 722 proteins and 4363.5 unique peptides (300 μg load analyses) were identified based on the methods described herein (Table 5). The present analyses identified more unique peptides and proteins than the best results (2725 peptides for 602 proteins) reported for HCPs in RM8671 NIST antibody, further confirming the high sensitivity and robustness of the present method for HCP analysis. In addition, an average of 90% (30 μg load) and 88% (300 μg load) proteins were common in process replicate analyses, showing the high reproducibility of the analyses. As expected, >93% of the proteins from 30 μg load analyses were also found in the analysis with 300 μg load, confirming the higher sensitivity of the 300 μg load analysis. The additional proteins identified from 300 μg load analysis compared to the 30 μg load analysis are likely to be present at levels between 0.1 and 1 ppm.

TABLE 5
Summary of identified HCPs in RM8671 NIST
antibody at different sample loads.

| | | | Protein | | |
|---|---|---|---|---|---|
| | | Average | Overlap | | |
| Sample | Unique | Protein | (from each | Average | Unique |
| Analysis | Proteins | Identifications | Rep.) | Overlap | Peptides |
| 30 μg Load | 618 | 599.5 | 514 (87%) | 90% | 3685 |
| Rep.1 | | | | | |
| 30 μg Load | 581 | | 514 (93%) | | 3494 |
| Rep.2 | | | | | |
| 300 μg Load | 746 | 722 | 623 (85%) | 88 | 4456 |
| Rep.1 | | | | | |
| 300 μg Load | 698 | | 623 (91%) | | 4271 |
| Rep.2 | | | | | |

At 30 and 300 μg sample loads, the present study identified 58 to 59 out of 60 proteins reported for RM 8670 NIST mAb in the original native digestion method. It was identified in the present disclosure an average of 421 (˜70%) and 466.5 (˜77%) proteins out of the reported 602 proteins, from 30 and 300 μg load analyses respectively. In addition, 95% of the top 199 (>5 peptide counts each) of the 602 proteins were also found by our 300 μg load analyses. The difference in protein identifications were likely due to the method differences; previous report applied additional Protein A depletion and FAIMS gas phase separation to the native digest conditions.

Through the evaluation of different aspects of the native digestion LC-MS/MS workflow, the present method shows the highest sensitivity (0.1 ppm) for HCP detection by DDA method and the highest HCP coverage (a total of 845 proteins) for RM8671NIST mAb. In contrast to previous studies that identified enzyme ratios at a 5 g/L protein concentration at digestion, the present study showed the advantage of higher protein concentration in identifying and quantitating low level HCPs. The present study is also the first work to show a predictable digestion condition can be established for representative drug product samples with a broad range of concentrations (2.5 to 25 g/L). When combining this method with parallel reaction monitoring (PRM), the present study was able to identify and quantitate hydrolases presented at levels as low as 0.01 ppm (Table 7) in mAb-1 using the original purification process, such as LipA and SMPD1, which were not identified from DDA due to their extremely low levels. The levels of the target hydrolases decreased after a modification was made to the original purification process. The purification modification was designed to improve HCP removal by adding an additional purification step, and therefore the results show the utility of the present methods to monitor clearance of known target hydrolases to support bioprocess development.

TABLE 7
Hydrolases in mAb-1 (before and after modifications were
made to the Purification Process) were quantified using
standard-addition based parallel reaction monitoring (PRM)

| | | Quantitative results* (ppm) | |
|---|---|---|---|
| | | Original | Modified |
| | | Purification | Purification |
| | | Process, | Process, |
| Protein | Peptide (SEQ ID NO:) | mAb-1 | mAb-1 |
| LipA | VNVYTSHSPAGTSVQNLR | 0.0133 | ND |
| | (SEQ ID NO: 1) | | |
| | HWGQIAK | 0.0114 | ND |
| | (SEQ ID NO: 2) | | |
| PPT1 | EIPGIYVLSLEIGK | 0.0102 | ND |
| | (SEQ ID NO: 3) | | |
| | ETIPLQESTLYTEDR | 0.0692 | ND |
| | (SEQ ID NO: 4) | | |
| SMPD1 | VLFTALNYGLK | 0.0096 | ND |
| | (SEQ ID NO: 5) | | |
| | ALTTVTDLVR | 0.0128 | ND |
| | (SEQ ID NO: 6) | | |
| LPL | GLGDVDQLVK | 0.5978 | 0.1717 |
| | (SEQ ID NO: 7) | | |
| | SIHLFIDSLLNEENPSK | 0.4504 | 0.0961 |
| | (SEQ ID NO: 8) | | |
| PLBL2 | SVLLDAASGQLR | 0.2213 | 0.0162 |
| | (SEQ ID NO: 9) | | |
| | LALDGATWADIFK | 0.2196 | ND |
| | (SEQ ID NO: 10) | | |
| ASAH1 | NLVNAFVPSGK | 0.4232 | 0.0707 |
| | (SEQ ID NO: 11) | | |
| | LTVFTTLIDVTK | 0.6921 | 0.0923 |
| | (SEQ ID NO: 12) | | |
| LPLA2 | HPPVVLVPGDLGNQLEAK | ND | ND |
| | (SEQ ID NO: 13) | | |
| | ATQFPDGVDVR | ND | ND |
| | (SEQ ID NO: 14) | | |
| PLD3 | ALLSVVDSAR | ND | ND |
| | (SEQ ID NO: 15) | | |
| | SFLLSLAALR | ND | ND |
| | (SEQ ID NO: 16) | | |

*Standard-addition based approach was used to monitor the hydrolase clearance before and after applying modifications to the purification process for mAb-1. First, a series of standard addition samples were created for each test sample by spiking in 0, 0.1, 0.5 and 1 ppm hydrolase standards respectively. Each standard addition sample was then subjected to the native digestion protocol described herein and subjected to PRM analysis with 2 top response peptides for each hydrolase. Data analysis was performed using Skyline software and standard addition plot for targeted peptides of each protein was then generated, with Spiked HCP level (ppm) as X-axis and the summed peak area of all fragment ions of targeted peptides as Y-axis. For each function Y = mX + b, the ppm level of target HCP in the test sample equals b/m, multiplied by percent purity of the original spiked protein stock standard if purity is <100%. When the 2 peptides of the protein showed different number, the highest level (bolded) was taken for the HCP quantitation. When the peptide was not detected, it was denoted by ND.

The present disclosure demonstrates an improved native-digest based workflow for sensitive and robust identification of residual HCPs at >0.1 ppm across broad range of protein sizes in purification in-process pool, drug substance or drug product samples for recombinant mAb therapeutics. It was found in the present work that a solid phase extraction (SPE) step is valuable to ensure more removal of undigested mAb from peptides for LC-MS analysis robustness. The present disclosure also demonstrated the effectiveness of SDC addition, improved bovine trypsin-to-protein ratio and high protein concentration (25 g/L) at digestion on recovery, identification, and quantitation of low levels of residual HCPs. With this improved workflow, native digest from up to about 300 μg starting material can be applied for sensitive and robust HCP analysis. It was also shown that the methods described herein can be modulated to achieve desired sensitivities ranging from 0.1 to 5 ppm by proportionally adjusting the amount of sample load. This modulated strategy allows the use of a single simple and flexible assay to address a broad range of HCP assay needs, ranging from safety risk assessment to bioprocess development. Furthermore, the workflow can be used either with DDA for unknown protein identification, or with targeted analysis for protein of interest with even higher sensitivity (0.01 ppm).

All publications, patents and other references cited herein are incorporated by reference in their entirety into the present disclosure.

1. A method for identifying one or more proteins in a sample comprising a protein product, comprising:
a) contacting a sample comprising one or more proteins with a protease under conditions sufficient to digest proteins present in the sample;
b) contacting the sample comprising the digested protein with sodium deoxycholate (SDC) under reducing and heated conditions;
c) contacting the sample comprising the digested protein to a chromatographic support to remove undigested protein;
d) contacting the chromatographic support with a mobile phase and collecting an eluent; and
e) analyzing a fraction of the eluent using liquid chromatography/mass spectrometry analysis (LC-MS/MS) to identify one or more proteins in the sample.

2. The method of claim 1 wherein the LC-MS/MS is performed in data dependent acquisition (DDA) mode.

3. The method of claim 1, wherein the protein is a host cell protein.

4.-6. (canceled)

7. The method of claim 1, wherein the w/w ratio of protease-to-protein product in the sample is about 1:2000, about 1:800, about 1:400, or about 1:200.

8. The method of claim 1, wherein the digested protein sample is contacted with SDC at about 0.9% w/v to about 1% w/v.

9.-10. (canceled)

11. The method of claim 1, wherein the method has a limit of detection (LOD) from about 0.1 ppm to about 5 ppm.

12. The method of claim 1, wherein the protein product is an antibody.

13. (canceled)

14. The method of claim 1, wherein the sample comprising the protein product is a partially or fully purified sample.

15. The method of claim 14, wherein the partially or fully purified sample is:
(i) an ultrafiltration/diafiltration pool sample,
(ii) a drug substance sample; or
(iii) a drug product sample.

16.-17. (canceled)

18. The method of claim 1, wherein a load of the protein product in the sample is from about 6 μg to about 300 μg.

19. The method of claim 1, wherein:
(i) the contacting step (a) is for a time from about 2 to about 4 hours and/or
(ii) the contacting step (a) is performed at about 37° C.; and/or
(iii) the contacting step (b) is for about 10 minutes; and/or
(iv) the contacting step (b) is performed at about 90° C.

20.-22. (canceled)

23. A method for determining the ppm level of one or more target proteins in a sample comprising a protein product, comprising:
a) contacting the sample with a protease under conditions sufficient to digest the one or more target proteins;
b) contacting the sample comprising the one or more digested target proteins with SDC under reducing and heated conditions;
c) contacting the sample comprising the one or more digested target proteins to a chromatographic support to remove undigested protein;
d) contacting the chromatographic support with a mobile phase and collecting an eluent; and
e) analyzing the eluent using LC-MS/MS to identify and quantitate the one or more target protein in the sample, wherein said analysis comprises determining the signals associated with a plurality of standard ppm levels of the one or more target protein and comparing those signals to the signal detected for the one or more target protein in the sample.

24. The method of claim 23, wherein the LC-MS/MS is performed in parallel reaction monitoring (PRM) mode.

25.-28. (canceled)

29. The method of claim 23, wherein the w/w ratio of protease-to-protein product in the sample is about 1:2000, about 1:800, about 1:400, or about 1:200.

30. The method of claim 23, wherein the one or more digested target protein sample is contacted with SDC at about 0.9% w/v.

31.-32. (canceled)

33. The method of claim 23, wherein the method has a limit of quantitation (LOQ) of about 0.01 ppm.

34. The method of claim 23, further comprising normalization of data from the LC-MS/MS analysis.

35. The method of claim 23, wherein the protein product is an antibody.

36. (canceled)

37. The method of claim 23, wherein the sample comprising the protein product is a partially or fully purified sample.

38. The method of claim 37, wherein the partially or fully purified sample is:
(i) a ultrafiltration/diafiltration pool sample;
(ii) a drug substance sample; or
(iii) a drug product sample.

39. (canceled)

40. The method of claim 23, wherein a load of the protein product in the sample is from about 6 μg to about 300 μg.

41. The method of claim 23, wherein:
(i) the contacting step (a) is for a time from about 2 to about 4 hours;
(ii) the contacting step (a) is performed at about 37° C.; and/or
(iii) the contacting step (b) is for about 10 minutes; and/or.
(iv) the contacting step (b) is performed at about 90° C.

42.-66. (canceled)

67. The method of claim 11, further comprising adjusting the sample load of the protein product to achieve the LOD from about 0.1 ppm to about 5 ppm.

68. The method of claim 1, wherein the fraction of the eluent contains about 6 μg, about 30 μg, about 60 μg, about 150 μg, or about 300 μg of protein product.

69. The method of claim 1, wherein analyzing step (e) further comprises normalization of data from the LC/MS analysis.


SEQUENCE LISTING (16 sequences)

SEQ ID NO: 1 - protein, 18 aa, synthetic construct
  REGION 1..18: note = Description of sequence: LipA peptide
VNVYTSHSPAGTSVQNLR

SEQ ID NO: 2 - protein, 7 aa, synthetic construct
  REGION 1..7: note = Description of sequence: LipA peptide
HWGQIAK

SEQ ID NO: 3 - protein, 14 aa, synthetic construct
  REGION 1..14: note = Description of sequence: PPT1 peptide
EIPGIYVLSLEIGK

SEQ ID NO: 4 - protein, 15 aa, synthetic construct
  REGION 1..15: note = Description of sequence: PPT1 peptide
ETIPLQESTLYTEDR

SEQ ID NO: 5 - protein, 11 aa, synthetic construct
  REGION 1..11: note = Description of sequence: SMPD1 peptide
VLFTALNYGLK

SEQ ID NO: 6 - protein, 10 aa, synthetic construct
  REGION 1..10: note = Description of sequence: SMPD1 peptide
ALTTVTDLVR

SEQ ID NO: 7 - protein, 10 aa, synthetic construct
  REGION 1..10: note = Description of sequence: LPL peptide
GLGDVDQLVK

SEQ ID NO: 8 - protein, 17 aa, synthetic construct
  REGION 1..17: note = Description of sequence: LPL peptide
SIHLFIDSLLNEENPSK

SEQ ID NO: 9 - protein, 12 aa, synthetic construct
  REGION 1..12: note = Description of sequence: PLBL2 peptide
SVLLDAASGQLR

SEQ ID NO: 10 - protein, 13 aa, synthetic construct
  REGION 1..13: note = Description of sequence: PLBL2 peptide
LALDGATWADIFK

SEQ ID NO: 11 - protein, 11 aa, synthetic construct
  REGION 1..11: note = Description of sequence: ASAH1 peptide
NLVNAFVPSGK

SEQ ID NO: 12 - protein, 12 aa, synthetic construct
  REGION 1..12: note = Description of sequence: ASAH1 peptide
LTVFTTLIDVTK

SEQ ID NO: 13 - protein, 18 aa, synthetic construct
  REGION 1..18: note = Description of sequence: LPLA2 peptide
HPPVVLVPGDLGNQLEAK

SEQ ID NO: 14 - protein, 11 aa, synthetic construct
  REGION 1..11: note = Description of sequence: LPLA2 peptide
ATQFPDGVDVR

SEQ ID NO: 15 - protein, 10 aa, synthetic construct
  REGION 1..10: note = Description of sequence: PLD3 peptide
ALLSVVDSAR

SEQ ID NO: 16 - protein, 10 aa, synthetic construct
  REGION 1..10: note = Description of sequence: PLD3 peptide
SFLLSLAALR